(19) United States
(12) Patent Application Publication
(10) Pub. No.: US 2008/0176898 A1
(43) Pub. Date: Jul. 24, 2008

(54) Phenyl Acetamides

(75) Inventors: Michael Harter, Leverkusen (DE); Frank Wunder, Wuppertal (DE); Lars Barfacker, Oberhausen (DE); Gunter Karig, Koln (DE); Walter Hubsch, Wuppertal (DE); Hanna Tinel, Wuppertal (DE); Eva-Maria Becker, Wuppertal (DE)

Correspondence Address: Bayer Health Care LLC, 400 Morgan Lane, West Haven, CT, 06516, US

(73) Assignee: Bayer HealthCare AG, Leverkusen (DE)

(21) Appl. No.: 11/587,318

(22) PCT Filed: Apr. 14, 2005
(86) PCT No.: PCT/EP2005/003926

§ 371 Date: Jan. 16, 2008

(30) Foreign Application Priority Data
Apr. 22, 2004 (DE) 102004019472.6

(51) Int. Cl. A61K 31/445; C07D 211/06; A61K 31/195; A61P 9/00; C07C 229/06

(52) U.S. Cl. 514/315; 546/342; 560/19; 514/563

(57) ABSTRACT

The present invention relates to phenylacetamides, to a process for their preparation and to their use for producing medicaments for the treatment and/or prophylaxis of diseases in humans and animals, especially of cardiovascular disorders.

The present invention relates to phenylacetamides, to a process for their preparation and to their use for producing medicaments for the treatment and/or prophylaxis of diseases in humans and animals, especially of cardiovascular disorders.

Cysteinyl leukotrienes are important mediators for a large number of pathological disease states. They are formed from arachidonic acid with the aid of 5-lipoxygenase on activation of inflammatory cells such as, for example, polymorphonuclear leukocytes, macrophages and mast cells. This involves initial production of leukotriene A4 (LTA4) which is then converted in further reaction steps by addition of glutathione into leukotriene C4 (LTC4). Further metabolism then results in leukotriene D4 (LTD4) and leukotriene E4 (LTE4). LTC4, LTD4 and LTE4 are together referred to as cysteinyl leukotrienes.

The physiological effects of cysteinyl leukotrienes are mediated by G protein-coupled receptors. Two cysteinyl leukotriene receptors have been characterized pharmacologically and molecular-biologically:

The cysteinyl leukotriene receptor 1 (CysLT1) is activated predominantly by LTD4, and more weakly by LTC4 and LTE4. It is therefore also referred to as the LTD4 receptor. The receptor was cloned and characterized in 1999 (Lynch et. al. (1999) Nature 399; 789-793). The CysLT1 receptor shows strong expression in the spleen, peripheral leukocytes and lung. CysLT1-specific receptor antagonists such as, for example, pranlukast, zafirlukast and montelukast lead to relaxation of the smooth muscles of the bronchi and have been developed for the treatment of bronchial asthma.

The cysteinyl leukotriene receptor 2 (CysLT2) is activated predominantly by LTC4, and more weakly by LTD4 and LTE4. It is therefore also referred to as LTC4 receptor. The receptor was identified and characterized in 2000 (Heise et. al. (2000) Journal of Biological Chemistry 275; 30531-30536; Takasaki et. al. (2000) Biochem. Biophys. Res. Commun. 274; 316-322; Nothacker et. al. (2000) Mol. Pharmacol. 58; 1601-1608). The human CysLT2 receptor shows very strong expression in the heart, placenta, spleen and peripheral blood leukocytes (PBL). It was possible to show by means of PCR investigations and in situ hybridizations that in the heart this receptor is expressed in smooth muscle cells of the coronary arteries, in myocytes and very strongly also in Purkinje fibers (Kamohara et. al. (2001) Biochem. Biophys. Res. Commun. 287; 1088-1092; Hui et. al. (2001) Journal of Biological Chemistry 276; 47489-47495). With an activation of the CysLT2 receptor there is, as with the CysLT1 receptor, an increase in the intracellular calcium concentration.

Cysteinyl leukotrienes are vasoactive substances, i.e. they lead to a strong constriction of coronary arteries. In addition, they reduce the contractility of the heart, induce changes in the electrocardiogram, influence the blood pressure, raise the microvascular permeability, promote the formation of edema and induce strong bronchoconstriction (Letts et. al (1987) Cardiovasc. Clin. 18; 101-113; Fauler and Frölich (1989) Cardiovasc. Drugs and Therapy 3; 499-505; Piper et. al. (1990) Adv. Prostaglandin Thromboxane Leukotr. Res. 20; 146-152). Antagonists of cysteinyl leukotriene receptors therefore form a therapeutic approach to the treatment of cardiovascular disorders.

EP-A 516 069, U.S. Pat. No. 4,826,990, U.S. Pat. No. 4,942,236 and U.S. Pat. No. 5,103,014 describe leukotriene antagonists for the treatment of allergic and antiinflammatory disorders. EP-A 791 576 and EP-A 341 551 disclose leukotriene antagonists for the treatment of asthma. Structurally similar structures are moreover described in U.S. Pat. No. 5,298,652 as phospholipase A2 inhibitors for the treatment of myocardial ischemia and in WO 95/32710 as products for inhibiting the resorption of bone mediated by osteoclasts.

The present invention relates to compounds of the formula

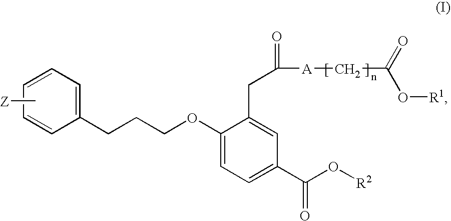

in which
A is a 4- to 7-membered nitrogen-containing saturated heterocycle which is bonded via the nitrogen atom to the keto group,
   or
   is a radical

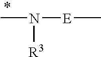

in which
E is (C₄-C₇)-cycloalkanediyl or (C₁-C₆)-alkanediyl,
R³ is hydrogen or methyl and
* is the point of linkage to the keto group,
n is 0, 1 or 2,
R¹ is hydrogen or (C₁-C₆)-alkyl,
R² is hydrogen or (C₁-C₆)-alkyl,
and
Z is located in the meta or para position and
   is a radical *-G-L-M-R⁴
   in which
   G is O,
   L is (C₁-C₆)-alkanediyl,
   M is a bond or O,
   R⁴ is (C₆-C₁₀)-aryl, biphenyl, phenoxyphenyl, benzyloxyphenyl, (E)-phenylvinylphenyl, 2-phenylethylphenyl, tetrahydronaphthyl, benzyl, heteroaryl, 5- to 10-membered heterocyclyl, (C₃-C₇)-cycloalkyl or (C₃-C₇)-cycloalkylmethyl, where aryl, biphenyl, phenoxyphenyl, benzyloxyphenyl, (E)-phenylvinylphenyl, 2-phenylethylphenyl, tetrahydronaphthyl, benzyl, heteroaryl, heterocyclyl, cyclo-alkyl and cycloalkylmethyl in turn may be substituted up to three times independently of one another by halogen, cyano, nitro, trifluoromethyl, trifluoromethoxy, (C₁-C₆)-alkyl, (C₁-C₆)-alkoxy, (C₂-C₆)-alkenyl, (C₃-C₇)-cyclo-alkyl, (C₃-C₇)-cycloalkylmethoxy, (C₅-C₇)-cycloalkenyl, (C₃-C₇)-cycloalkoxy or (C₅-C₇)-cycloalkenyloxy, and
   is the point of linkage to the phenyl ring,

and the salts, hydrates, hydrates of the salts and solvates thereof.

Compounds according to the invention are the compounds of the formula (I) and the salts, solvates and solvates of the salts thereof, the compounds of the formulae mentioned below which are encompassed by formula (I), and the salts, solvates and solvates of the salts thereof, and the compounds encompassed by formula (I) and mentioned below as exemplary embodiments, and the salts, solvates and solvates of the salts thereof, insofar as the compounds encompassed by formula (I) and mentioned below are not already salts, solvates and solvates of the salts.

The compounds of the invention may, depending on their structure, exist in stereoisomeric forms (enantiomers, diastereomers). The invention therefore relates to the enantiomers or diastereomers and respective mixtures thereof. The stereoisomerically pure substituents can be isolated in a known manner from such mixtures of enantiomers and/or diastereomers.

Where the compounds of the invention can occur in tautomeric forms, the present invention encompasses all tautomeric forms.

Salts preferred for the purposes of the present invention are physiologically acceptable salts of the compounds of the invention. However, salts which are themselves unsuitable for pharmaceutical applications but can be used for example for isolating or purifying the compounds of the invention are also encompassed.

Physiologically acceptable salts of the compounds of the invention include acid addition salts of mineral acids, carboxylic acids and sulfonic acids, e.g. salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, trifluoroacetic acid, propionic acid, lactic acid, tartaric acid, malic acid, citric acid, fumaric acid, maleic acid and benzoic acid.

Physiologically acceptable salts of the compounds of the invention also include salts of conventional bases such as, for example and preferably, alkali metal salts (e.g. sodium and potassium salts), alkaline earth metal salts (e.g. calcium and magnesium salts) and ammonium salts derived from ammonia or organic amines having 1 to 16 C atoms, such as, for example and preferably, ethylamine, diethylamine, triethylamine, ethyldiisopropylamine, monoethanolamine, diethanolamine, triethanolamine, dicyclohexylamine, dimethylaminoethanol, procaine, dibenzylamine, N-methyl-morpholine, arginine, lysine, ethylenediamine and N-methylpiperidine.

Solvates refer for the purposes of the invention to those forms of the compounds of the invention which form a complex in the solid or liquid state through coordination with solvent molecules. Hydrates are a specific form of solvates in which the coordination takes place with water.

Alkyl per se and “alk” in alkoxy stand for a linear or branched alkyl radical having usually 1 to 6, preferably 1 to 4, particularly preferably 1 to 3, carbon atoms, by way of example and preferably methyl, ethyl, n-propyl, isopropyl, tert-butyl, n-pentyl and n-hexyl.

Alkoxy stands by way of example and preferably for methoxy, ethoxy, n-propoxy, isopropoxy, tert-butoxy, n-pentoxy and n-hexoxy.

Alkanediyl stands for a straight-chain or branched saturated alkanediyl radical having 1 to 6 carbon atoms. A straight-chain or branched alkanediyl having 1 to 4 carbon atoms is preferred. Preferred examples which may be mentioned are methylene, ethane-1,2-diyl, ethane-1,1-diyl, propane-1,3-diyl, propane-1,2-diyl, propane-2,2-diyl, butane-1,4-diyl, butane-1,3-diyl, butane-2,4-diyl, pentane-1,5-diyl, pentane-2,4-diyl, 2-methyl-pentane-2,4-diyl.

Alkenyl stands for a straight-chain or branched alkenyl radical having 2 to 6 carbon atoms. A straight-chain or branched alkenyl radical having 2 to 4 carbon atoms is preferred. Preferred examples which may be mentioned are: vinyl, allyl, n-prop-1-en-1-yl, n-but-2-en-1-yl and 2-methyl-2-buten-1-yl.

Cycloalkyl per se and “cycloalk” in cycloalkoxy and cycloalkanediyl stands for a cycloalkyl group having usually 3 to 8, preferably 5 to 7, carbon atoms, by way of example and preferably cyclopropyl, cyclobutyl, cyclopentyl, cyclohexyl and cycloheptyl.

Cycloalkoxy stands by way of example and preferably for cyclopropyloxy, cyclobutyloxy, cyclopentyloxy, cyclohexyloxy and cycloheptyloxy.

Cycloalkanediyl stands by way of example and preferably for cyclopropane-1,2-diyl, cyclobutane-1,2-diyl, cyclobutane-1,3-diyl, cyclopentane-1,2-diyl, cyclopentane-1,3-diyl, cyclohexane-1,2-diyl, cyclohexane-1,3-diyl, cyclohexane-1,4-diyl, cycloheptane-1,2-diyl, cycloheptane-1,3-diyl and cycloheptane-1,4-diyl.

Cycloalkenyl per se and “cycloalken” in cycloalkenyloxy stands for a cycloalkenyl group having usually 5 to 7 carbon atoms, by way of example and preferably cyclopent-2-en-1-yl, cyclopent-3-en-1-yl, cyclohex-2-en-1-yl, cyclohex-3-en-1-yl, cyclohept-2-en-1-yl, cyclohept-3-en-1-yl and cyclohept-4-en-1-yl.

Cycloalkenyloxy stands by way of example and preferably for cyclopent-2-en-1-yloxy, cyclopent-3-en-1-yloxy, cyclohex-2en-1-yloxy, cyclohex-3-en-1-yloxy, cyclohept-2-en-1-yloxy, cyclohept-3-en-1-yloxy and cyclohept-4-en-1-yloxy.

Aryl stands for a mono- to tricyclic aromatic, carbocyclic radical having usually 6 to 10 carbon atoms; by way of example and preferably phenyl and naphthyl.

Heteroaryl stands for an aromatic, mono- or bicyclic, optionally benzo-fused, radical having usually 5 to 10, preferably 5 to 6, ring atoms and up to 5, preferably up to 4, heteroatoms from the series S, O and N, by way of example and preferably thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl, isoquinolinyl, benzimidazolyl and benzoxazolyl.

Heterocyclyl stands for a mono- or polycyclic, preferably mono- or bicyclic, optionally benzo-fused, nonaromatic heterocyclic radical having usually 5 to 10, preferably 5 to 8, in particular 5 or 6, ring atoms and up to 3, preferably up to 2, heteroatoms and/or hetero groups from the series N, O, S, SO, SO₂. The heterocyclyl radicals may be saturated or partially unsaturated. 5- or 6-membered, monocyclic saturated heterocyclyl radicals having up to two heteroatoms from the series O, N and S, which may optionally be benzo-fused, are preferred, such as by way of example and preferably tetrahydrofuran-2-yl, pyrrolidin-2-yl, pyrrolidin-3-yl, pyrrolinyl, piperidinyl, morpholinyl, perhydroazepinyl, 1,3-benzodioxolyl, tetrahydro-2H-pyranyl, 1,3-dioxanyl, 1,4-dioxanyl, 2,3-dihydro-1,4-dioxinyl, 2,3-dihydro-1,4-benzodioxinyl and 4H-1,3-benzodioxinyl.

Halogen stands for fluorine, chlorine, bromine and iodine.

If radicals in the compounds of the invention are substituted, the radicals may, unless specified otherwise, have one or more identical or different substituents. Substitution by up to three identical or different substituents is preferred, substitution by one substituent is particularly preferred,

Preference is given to compounds of the formula (I), in which
A is a 4- to 6-membered nitrogen-containing saturated heterocycle which is bonded via the nitrogen atom to the keto group,
   or
   is a radical

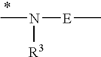

in which
E is cyclopentanediyl, cyclohexanediyl, methylene, ethane-1,2-diyl or propane-1,3-diyl,
R³ is hydrogen, and
* is the point of linkage to the keto group,
n is 0 or 1,
R¹ is hydrogen,
R² is hydrogen,
and
Z is located in the meta or para position and
   is a radical *-G-L-M-R⁴
   in which
   G is O,
   L is (C₁-C₄)-alkanediyl,
   M is a bond or O,
   R⁴ is phenyl, naphthyl, biphenyl, phenoxyphenyl, benzyloxyphenyl, (E)-phenylvinylphenyl, 2-phenylethylphenyl, tetrahydronaphthyl, benzyl, 1,3-dioxanyl, 1,4dioxanyl, dimethyl-1,3-dioxanyl, tetrahydro-2H-pyranyl, (C₃-C₇)-cycloalkyl or (C₃-C₇)-cycloalkylmethyl, where phenyl, naphthyl, biphenyl, phenoxyphenyl, benzyloxyphenyl, (E)-phenylvinylphenyl, 2-phenylethylphenyl, tetrahydronaphthyl, benzyl, cycloalkyl and cycloalkylmethyl in turn may be substituted up to three times independently of one another by halogen, cyano, nitro, trifluoromethyl, trifluoromethoxy, (C₁-C₆)-alkyl, (C₁-C₆)-alkoxy, (C₃-C₇)-cycloalkyl, (C₃-C₇)-cyclo-alkylmethoxy or (C₃-C₇)-cycloalkoxy, and
   * is the point of linkage to the phenyl ring,

and the salts, hydrates, hydrates of the salts and solvates thereof.

Particular preference is given to compounds of the formula (I) in which
A is acetidine, pyrrolidine or piperidine which is bonded via the nitrogen atom to the keto group,
   or
   is a radical

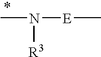

in which
E is cyclopentanediyl, cyclohexanediyl, methylene, ethane-1,2-diyl or propane-1,3-diyl,
R³ is hydrogen, and
* is the point of linkage to the keto group,
n is 0 or 1,
R¹is hydrogen,
R² is hydrogen,
and
Z is located in the para position and
   is a radical *-G-L-M-R⁴
   in which
   G is O,
   L is methylene, propane-1,3-diyl or butane-1,4-diyl,
   M is a bond or O,
   R⁴ is phenyl, 4-biphenyl, 4-phenoxyphenyl, 4-benzyloxyphenyl, 1,2,3,4-tetrahydronaphth-6-yl, 5,5-dimethyl-1,3-dioxan-2-yl or cyclohexyl, where phenyl in turn may be substituted by halogen, trifluoromethoxy, (C₃-C₄)-alkyl, (C₃-C₄)-alkoxy, cyclopentyl, cyclohexyl or (C₃-C₆)-cycloalkylmethoxy, and
   * is the point of linkage to the phenyl ring,

and the salts, hydrates, hydrates of the salts and solvates thereof.

Very particular preference is given to compounds of the formula (I) in which
A-[CH₂]ₙ—CO₂R¹ is a radical

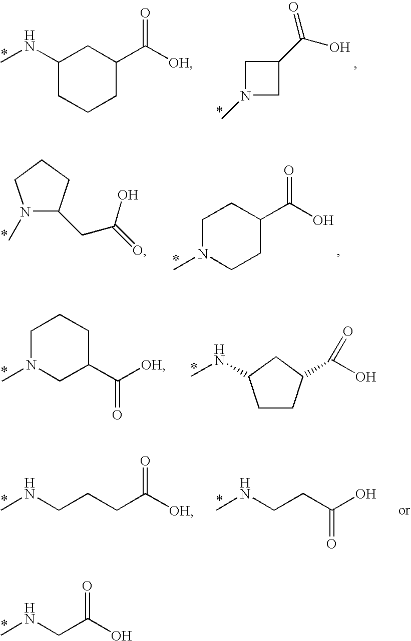

in which
* is the point of linkage to the keto group,
R² is hydrogen,
and
Z is located in the para position and is
   a radical *—O—CH₂—R⁴,
   in which
   R⁴ is phenyl, 4-biphenyl, 4-phenoxyphenyl, 4-benzyloxyphenyl or 1,2,3,4-tetrahydronaphth-6-yl, where phenyl in turn may be substituted by trifluoromethoxy, n-propyl, n-butyl, tert-butyl, n-propyloxy, isopropyloxy, isobutyloxy, cyclohexyl or cyclopropylmethoxy, and
   * is the point of linkage to the phenyl ring,
   or
   a radical *—O—CH₂—CH₂—CH₂—R⁴
   in which
   R⁴ is 4-chlorophenyl, 5,5-dimethyl-1,3-dioxan-2-yl or cyclohexyl, and
   * is the point of linkage to the phenyl ring,
   or
   is a radical *—O—CH₂—CH₂—CH₂—CH₂—O—R₄
   in which
   R⁴ is phenyl or cyclohexyl, and
   is the point of linkage to the phenyl ring,

and the salts, hydrates, hydrates of the salts and solvates thereof.

Very particular preference is also given to compounds of the formula (I) in which
A is acetidine, pyrrolidine or piperidine which is bonded via the nitrogen atom to the keto group,
   or
   is a radical

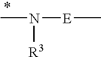

in which
E is cyclopentanediyl, cyclohexanediyl, methylene, ethane-1,2-diyl or propane-1,3-diyl,
R³ is hydrogen, and
* is the point of linkage to the keto group,
n is 0 or 1,
R¹ is hydrogen,
R² is hydrogen,
and
Z is located in the para position and is a radical

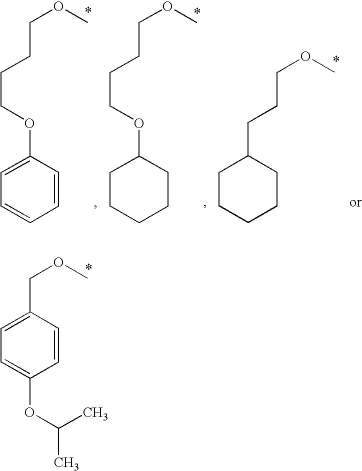

in which
* is the point of linkage to the phenyl ring,

and the salts, hydrates, hydrates of the salts and solvates thereof.

Especially preferred are compounds of the formula (I) in which
A-[CH₂]ₙ—CO₂R¹ is a radical

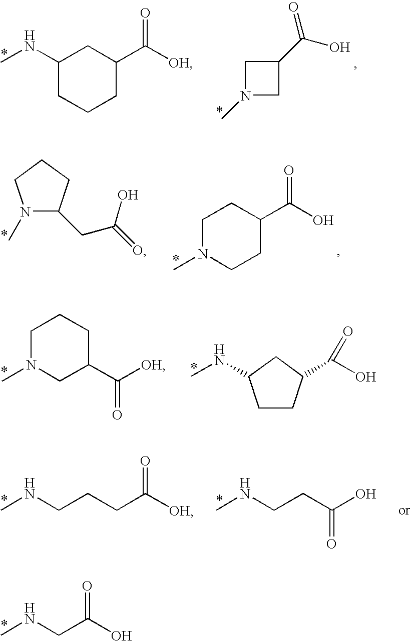

in which
* is the point of linkage to the keto group,
R² is hydrogen,
and
Z is located in the para position and is a radical

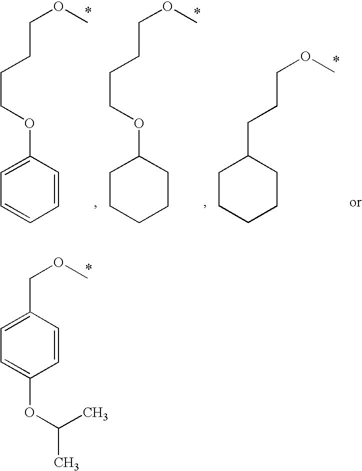

in which
* is the point of linkage to the phenyl ring,

and the salts, hydrates, hydrates of the salts and solvates thereof.

The present invention further relates to a process for preparing the compounds of the formula (I) which is characterized in that
either
[A] compounds of the formula

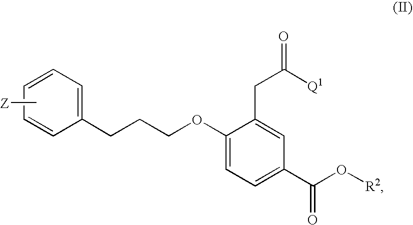

in which
R² is (C₁-C₆)-alkyl,
Q¹ is hydroxy or chlorine, and
Z has the meaning indicated above,
are reacted with compounds of the formula

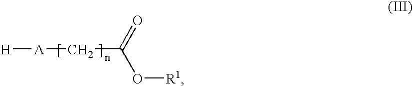

in which
R¹ is (C₁-C₆)-alkyl, and
n and A have the meaning indicated above,
or
[B] the two ester groups —C(O)OR¹ and —C(O)OR² in compounds prepared by process step [A] are hydrolyzed.
Compounds of the formula

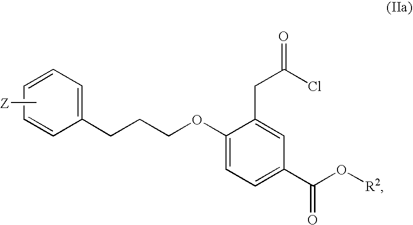

in which
R² is (C₁-C₆)-alkyl, and
Z has the meaning indicated above,
can be prepared for example by converting the carboxylic acid group in compounds of the formula

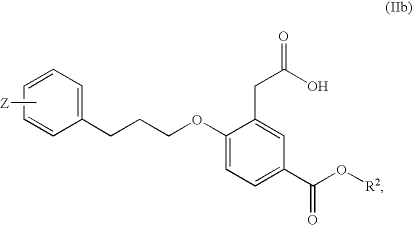

in which
R² is (C₁-C₆)-alkyl, and
Z has the meaning indicated above,

into the corresponding acid chloride by chlorination.

Formula (II) encompasses the compounds of the formulae (IIa) and (IIb).

Compounds of the formula (IIb) can be prepared for example by reacting compounds of the formula

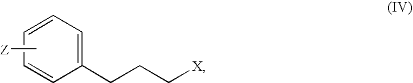

in which
X is a suitable leaving group such as, for example, halogen, mesylate or tosylate, and
Z has the meaning indicated above,

with compounds of the formula

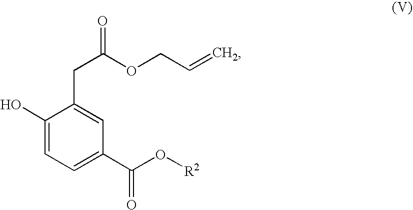

in which
R² is (C₁-C₆)-alkyl,

to give compounds of the formula

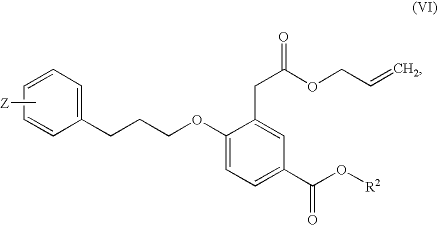

in which
R² is (C₁-C₆)-alkyl, and
Z has the meaning indicated above,

and subsequently eliminating the allyl group.

Compounds of the formula (IV) can be prepared for example by converting compounds of the formula

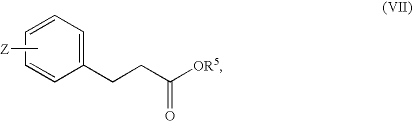

in which
R⁵ is hydrogen or an alkyl radical, and
Z has the meaning indicated above,

by reduction of the carboxylic acid or ester group into the corresponding alcohols of the formula

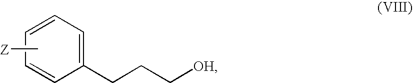

in which
Z has the meaning indicated above,

and finally converting the hydroxy group into a leaving group such as, for example, halogen, mesylate or tosylate.

The compounds of the formula (V) can be prepared for example in analogy to P. Nussbaumer et al., J. Org. Chem. 2000, 65, 7660-7662.

The compounds of the formula (III) and (VII) are known per se to the skilled worker or can be prepared by conventional processes disclosed in the literature.

In the amide coupling of process step (II)+(III)→(I) (for Q¹ equal to hydroxy), the amines are preferably employed in the form of their hydrochlorides. The reactions preferably take place under standard conditions in the presence of generally customary reagents for amide or peptide coupling, such as, for example, N-[(3-dimethylamino)propyl]-N′-ethylcarbodiimide hydrochloride (EDC) and 1-hydroxy-1H-benzotriazole hydrate (HOBT) in the presence of auxiliary bases such as triethyl-amine or diisopropylethylamine, in solvents such as dichloromethane or dimethylformamide at room temperature.

In the amide coupling of process step (II)+(III)→(I) (for Q¹ equal to chlorine), the amines are preferably employed in the form of their hydrochlorides. The reactions preferably take place under standard conditions in the presence of auxiliary bases such as triethylamine or diisopropylethylamine, in solvents such as diethyl ether, tetrahydrofuran or methylene chloride at temperatures between 0° C. and room temperature.

The ester hydrolysis in the preparation of compounds (I) by process [B] in which R¹ and R² are then hydrogen preferably takes place in the presence of aqueous alkali metal hydroxide solution such as, for example, 2 molar sodium hydroxide solution at temperatures between room temperature and 70° C. with addition of water-miscible organic solvents such as, for example, methanol or tetrahydrofuran or mixtures thereof.

Process step (IIb)→(IIa) preferably takes place with chlorinating reagents such as thionyl chloride or oxalyl chloride. The reaction takes place where appropriate in the presence of catalytic amounts of dimethylformamide, it being possible to add halogenated hydrocarbons such as, for example, dichloromethane or chloroform as solvents. The reaction temperature in this case is between 0° C. and the boiling point of the respective solvent or chlorinating reagent.

Process step (IV)+(V)→(VI) preferably takes place in inert solvents such as, for example, tetrahydrofuran, dioxane, dimethylformamide, N-methylpyrrolidone, acetonitrile, acetone or butyronitrile, in the presence of auxiliary bases such as, for example, potassium carbonate, sodium carbonate, cesium carbonate, triethylamine, ethyldiisopropylamine or pyridine in a temperature range between room temperature and the boiling point of the respective solvent.

Elimination of the allyl group in process step (VI)→(IIb) takes place under conventional conditions known to the skilled worker for eliminating allyl groups, such as, for example, with palladium catalysts such as tetrakis(triphenylphosphine)palladium(0) in the presence of amine bases such as morpholine in inert solvents such as, for example, tetrahydrofuran in the temperature range between 0° C. and the boiling point of the respective solvent.

Process step (VII)→(VIII) takes place under usual conditions known to the skilled worker for reducing carboxylic or ester groups to the corresponding alcohol groups, such as, for example, with complex metal hydrides such as lithium aluminium hydride in inert solvents such as, for example, tetrahydrofuran in the temperature range between 0° C. and the boiling point of the respective solvent.

The conversion of the alcohol function into a leaving group X in process step (VIII)→(IV) can take place in various ways known to the skilled worker. The reaction to give the corresponding bromide preferably takes place in tetrahydrofuran as solvent at room temperature with a mixture of triphenylphosphine and tetrabromomethane or in dichloromethane as solvent in a temperature range between 0° C. and room temperature with phosphorus tribomide, where appropriate in the presence of pyridine. The reaction to give the corresponding mesylate or tosylate preferably takes place in dichloromethane as solvent in the temperature range between 0° C. and room temperature with methanesulfonyl chloride or para-toluenesulfonyl chloride in the presence of tertiary amines such as, for example, triethylamine or disopropylethylamine.

The process of the invention can be illustrated by way of example by the following formula scheme:

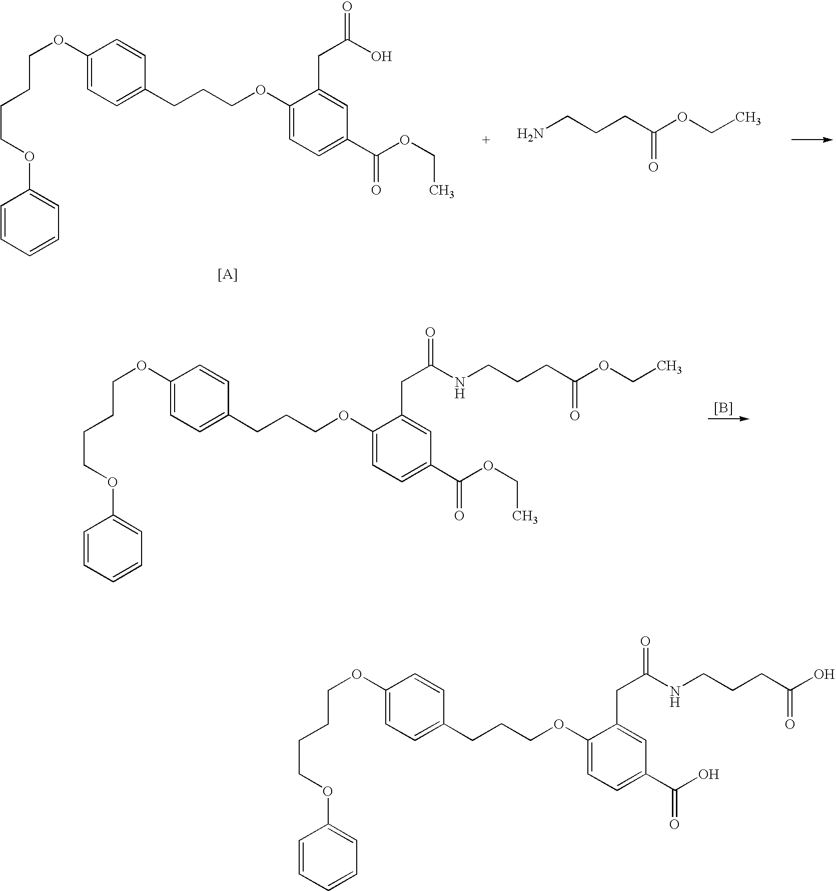

The compounds of the invention show a valuable pharmacological and pharmacokinetic spectrum of effects which could not have been predicted.

They are therefore suitable for use as medicaments for the treatment and/or prophylaxis of diseases in humans and animals.

The pharmaceutical activity of the compounds of the invention can be explained by their effect as selective antagonists of the cysteinyl-leukotriene receptor 2.

Cysteinyl-leukotriene receptor antagonists referred to as “selective” for the purposes of the present invention are those which inhibit the activity of the cysteinyl-leukotriene receptor 2 at a concentration which is lower by a factor of more than 10, preferably by a factor or more than 100, in particular by a factor of more than 1000, than an equivalent activity of the cysteinyl-leukotriene receptor 1. Concerning the test methods for determining the selectivity, reference may be made to the test methods described in section B 1. and B 2.

Modulators of the cysteinyl-leukotriene receptors referred to as “antagonists” for the purposes of the present invention are those having antagonistic activity and comprising merely a partial, preferably no measurable, agonistic component.

The compounds of the formula (I) are suitable for the prophylaxis and/or treatment of various disorders, preferably of cardiovascular disorders.

Preferred examples which may be mentioned are: atrial and ventricular arrhythmias, myocardial infarction, arteriosclerosis, heart failure, stable and unstable angina pectoris, myocardial ischemia, transient and ischemic attacks, stroke, inflammatory cardiovascular disorders, coronary heart disease, peripheral and cardiac vascular disorders, peripheral blood flow disturbances, restenoses such as following thrombolysis therapies, percutaneous transluminal angioplasties (PTA) and transluminal coronary angioplasties (PTCA), pulmonary hypertension, coronary spasms, thromboses, thromboembolic disorders, bypass operations, heart transplants, edema formation, shock, high blood pressure, acute renal failure, inflammatory disorders, asthmatic disorders, chronic obstructive airways disease (COPD), states of pain, prostate hypertrophy, inflammatory skin disorders, placental insufficiency, placentation disturbances, incontinence, cystitis, hyperactive bladder, disorders of the adrenal such as, for example, pheochromocytoma and Waterhouse-Friderichsen syndrome, intestinal disorders such as, for example, Crohn's disease or diarrhea.

The present invention further relates to the use of the compounds of the invention for the treatment and/or prophylaxis of disorders, in particular of the aforementioned pathological states.

The present invention further relates to the use of the compounds of the invention for producing a medicament for the treatment and/or prophylaxis of disorders, in particular of the aforementioned pathological states.

The present invention further relates to a method for the treatment and/or prophylaxis of disorders, in particular of the aforementioned disorders, using an amount having cardiovascular efficacy of the compound of the invention.

The present invention further relates to medicaments comprising a compound of the invention and one or more other active ingredients, in particular for the treatment and/or prophylaxis of the aforementioned disorders. Examples of suitable and preferred active ingredients in the combination which may be mentioned are: cysteinyl-leukotriene receptor 1 antagonist, cysteinyl-leukotriene-biosynthesis inhibitor, thrombolytics, a platelet aggregation inhibitor, β-blockers, nitrates, Ca channel blockers and/or an anti-inflammatory active ingredient such as, for example, a cyclooxygenase inhibitor.

The compound of the invention may have systemic and/or local effects. It can for this purpose be administered in a suitable way, such as, for example, by the oral, parenteral, pulmonary, nasal, sublingual, lingual, buccal, rectal, dermal, transdermal, conjunctival or otic route or as implant or stent.

The compound of the invention can be administered in suitable administration forms for these administration routes.

Administration forms suitable for oral administration are those which function according to the state of the art and deliver the compounds of the invention in a rapid and/or modified way, and which contain the compounds of the invention in crystalline and/or amorphized and/or dissolved form, such as, for example, tablets (uncoated or coated tablets, for example with coatings which are resistant to gastric juice or dissolve slowly or are insoluble and which control the release of the compound of the invention), tablets which rapidly disintegrate in the mouth, or films/wafers, capsules, sugar-coated tablets, granules, pellets, powders, emulsions, suspensions, aerosols or solutions.

Parenteral administration can take place with avoidance of an absorption step (e.g. intravenous, intraarterial, intracardiac, intraspinal or intralumbar) or with inclusion of an absorption (e.g. intramuscular, subcutaneous, intracutaneous, percutaneous or intraperitoneal). Administration forms suitable for parenteral administration are, inter alia, injection and infusion preparations in the form of solutions, suspensions, emulsions, lyophilisates or sterile powders.

Examples suitable for other administration routes are medicinal forms for inhalation (inter alia powder inhalators, nebulizers), nasal drops, solutions, sprays; tablets or capsules for lingual, sublingual or buccal administration, suppositories, preparations for the ears or eyes, vaginal capsules, aqueous suspensions (lotions, shaking mixtures), lipophilic suspensions, ointments, creams, milk, pastes, dusting powders, stents or implants.

Parenteral, in particular intravenous administration is preferred, e.g. as iv bolus injection (i.e. as single dose, e.g. by syringe), short infusion (i.e. infusion over a period of up to one hour) or long infusion (i.e. infusion over a period of more than one hour). The administered volume may in these cases be, depending on the specific conditions, between 0.5 to 30, in particular 1 to 20, ml on iv bolus injection, between 25 to 500, in particular 50 to 250, ml on short infusion and between 50 to 1000, in particular 100 to 500, ml on long infusion. It may for this purpose be advantageous for the active ingredient to be provided in the solid form (e.g. as lyophilisate or as salt) and to be dissolved in the dissolving medium only directly before administration.

It is necessary in these cases that the administration forms be sterile and pyrogen-free. They may be based on aqueous or mixtures of aqueous and organic solvents. These include, for example, aqueous solutions, mixtures of aqueous and organic solvents (especially ethanol, polyethylene glycol (PEG) 300 or 400), aqueous solutions containing cyclodextrins or aqueous solutions containing emulsifiers (surface-active solubilizers, e.g. lecithin or Pluronic F 68, Solutol HS15, Cremophor). Aqueous solutions are preferred.

Formulations suitable for parenteral administration are those which are substantially isotonic and euhydric, e.g. those with a pH between 3 and 11, preferably between 6 and 8, in particular around 7.4.

Injection solutions are packaged in suitable containers made of glass or plastic, e.g. in vials. The solution can be removed directly therefrom and administered. In the case of a lyophilisate, it is dissolved in the vial by injecting a suitable solvent and is then removed. Infusion solutions are packaged in suitable containers made of glass or plastic, e.g. in bottles or collapsible plastic bags.

The compounds of the invention can be converted into the stated administration forms in a manner known per se. This takes place by using inert, non-toxic, pharmaceutically suitable excipients. These excipients include, inter alia, carriers (for example microcrystalline cellulose), solvents (e.g. liquid polyethylene glycols), emulsifiers (for example sodium dodecyl sulfate), dispersants (for example biopolyvinylpyrrolidone), synthetic and natural polymers (for example albumin), stabilizers (e.g. antioxidants such as, for example, ascorbic acid), colors (e.g. inorganic pigments such as, for example, iron oxides) and masking tastes and/or odors.

The present invention further relates to medicaments which comprise at least one compound of the invention, preferably together with one or more inert, non-toxic, pharmaceutically suitable excipients, and to the use thereof for the aforementioned purposes.

It has generally proved advantageous both in human and in veterinary medicine to administer the compound of the invention in total amounts of about 0.01 to about 700, preferably 0.01 to 100, mg/kg of body weight every 24 hours, where appropriate in the form of a plurality of single doses, to achieve the desired results. A single dose preferably contains the compound of the invention in amounts of about 0.1 to about 80, in particular 0.1 to 30, mg/kg of body weight.

It may nevertheless be necessary to deviate from the stated amounts, in particular as a function of body weight, administration route, individual behavior towards the active ingredient, type of preparation and time or interval over which administration takes place. Thus, it may in some cases be sufficient to make do with less than the aforementioned minimum amount, whereas in other cases the stated upper limit must be exceeded. Where larger amounts are administered, it may be advisable to divide them into a plurality of single doses over the day.

The percentage data in the following tests and examples are, unless indicated otherwise, percentages by weight; parts are parts by weight. Solvent ratios, dilution ratios and concentration data for liquid/liquid solutions are in each case based on volume.

### A. EXAMPLES

Abbreviations:
b.p. boiling point
conc. concentrated
DCI direct chemical ionization (in MS)
DCM dichloromethane
DIEA N,N-diisopropylethylamine
DMSO dimethyl sulfoxide
DMF N,N-dimethylformamide
EA ethyl acetate (acetic acid ethyl ester)
EI electron impact ionization (in MS)
ESI electrospray ionization (in MS)
h hour
HPLC high pressure, high performance liquid chromatography
LC-MS coupled liquid chromatography/mass spectroscopy
LDA lithium diisopropylamide
m.p. melting point
MPLC medium pressure, medium performance liquid chromatography
MS mass spectroscopy
NMR nuclear magnetic resonance spectroscopy
RP-HPLC reverse phase HPLC
RT room temperature
R_{f} retention index (in TLC)
Rₜ retention time (in HPLC)
sat. saturated
TFA trifluoroacetic acid
THF tetrahydrofuran
TLC thin-layer chromatography

HPLC- and LC-MS methods:

Method 1 (HPLC): instrument HP 1100 with DAD detection; column: Kromasil RP-18, 60 mm×2.1 mm, 3.5 μm; eluent A: 5 ml of perchloric acid/l of water, eluent B: acetonitrile; gradient: 0 min 2% B, 0.5 min 2% B, 4.5 min 90% B, 6.5 min 90% B, 6.7 min 2% B, 7.5 min 2% B; flow rate: 0.75 ml/min; oven: 30° C.; UV detection: 210 nm.

Method 2 (HPLC): instrument HP 1100 with DAD detection; column: Kromasil RP-18, 60 mm×2.1 mm, 3.5 μm; eluent A: 5 ml of perchloric acid/l of water, eluent B: acetonitrile; gradient: 0 min 2% B, 0.5 min 2% B, 4.5 min 90% B, 9 min 90% B, 9.2 min 2% B, 10 min 2% B; flow rate: 0.75 ml/min; oven: 30° C.; Lw detection: 210 nm.

Method 3 (HPLC): instrument HP 1100 with DAD detection; column: Kromasil RP-18, 60 mm×2.1 mm, 3.5 μm; eluent A: 5 ml of perchloric acid/l of water, eluent B: acetonitrile; gradient: 0 min 2% B, 0.5 min 2% B, 4.5 min 90% B, 15 min 90% B, 15.2 min 2% B, 16 min 2% B; flow rate: 0.75 ml/min; oven: 30° C.; UV detection: 210 nm.

Method 4 (LC-MS): MS instrument type: Micromass ZQ; HPLC instrument type: HP 1100 series; UV DAD; column: Phenomenex Synergi 2μ Hydro-RP Mercury 20 mm×4 mm; eluent A: 1 l of water+0.5 ml of 50% formic acid, eluent B: 1 l acetonitrile+0.5 ml of 50% formic acid; gradient: 0.0 min 90% A→2.5 min 30% A→3.0 min 5% A→4.5 min 5% A; flow rate: 0.0 min 1 ml/min, 2.5 min/3.0 min/4.5 min. 2 ml/min; oven: 50° C.; UV detection: 210 nm.

Method 5 (LC-MS): MS instrument type: Micromass ZQ; HPLC instrument type: Waters Alliance 2795; column: Merck Chromolith SpeedROD RP-18e 50 mm×4.6 mm; eluent A: water+500 μl of 50% formic acid/l; eluent B: acetonitrile+500 μl of 50% formic acid/l; gradient: 0.0 min 10% B→3.0 min 95% B→+4.0 min 95% B; oven: 35° C.; flow rate: 0.0 min 1.0 ml/min→3.0 min 3.0 ml/min→4.0 min 3.0 ml/min; UV detection: 210 nm.

Method 6 (LC-MS): instrument Micromass Quattro LCZ, with HPLC Agilent series 1100; column: Grom-SIL120 ODS-4 HE, 50 mm×2.0 mm, 3 μm; eluent A: 1 l of water+1 ml of 50% formic acid, eluent B: 1 l acetonitrile+1 ml of 50% formic acid; gradient: 0.0 min 100% A→0.2 min 100% A→2.9 min 30% A→3.1 min 10% A→4.5 min 10% A; oven: 55° C.; flow rate: 0.8 ml/min; UV detection: 208-400 nm.

Method 7 (LC-MS): MS instrument type: Micromass ZQ; HPLC instrument type: Waters Alliance 2795; column: Phenomenex Synergi 2μ Hydro-RP Mercury 20 mm×4 mm; eluent A: 1 l of water+0.5 ml of 50% formic acid, eluent B: 1 l acetonitrile+0.5 ml 50% of formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 210 nm.

Method 8 (LC-MS): instrument Micromass Quattro LCZ with HPLC Agilent series 1100; column: Phenomenex Synergi 2μ Hydro-RP Mercury 20 mm×4 mm; eluent A: 1 l of water+0.5 ml of 50% formic acid, eluent B: 1 l acetonitrile+0.5 ml of 50% formic acid; gradient: 0.0 min 90% A→+2.5 min 30% A→+3.0 min 5% A→+4.5 min 5% A; flow rate: 0.0 min 1 ml/min, 2.5 min/3.0 min/4.5 min 2 ml/min; oven: 50° C.; UV detection: 208-400 nm.

Method 9 (HPLC): column: Symmetry TM C18 3.9 mm×150 mm; eluent A: water, eluent B: acetonitrile; gradient: 0.0 min 10% B→+0.6 min 10% B→+3.8 min 100% B→+5.0 min 100% B→5.5 min 10% B→6.0 min 10% B; flow rate: 1.5 ml/min; volume injected: 10 μl; UV detection: 214 nm and 254 nm.

Starting Compounds

### Example I

### Ethyl 4-hydroxy-3-(hydroxymethyl)benzoate

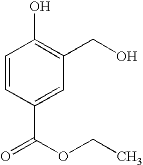

10.0 g (51.50 mmol) of ethyl 4-formyl-3-(hydroxymethyl)benzoate [CAS No. 82304-99-2] are introduced into 250 ml of ethanol and, at 0° C., solid sodium borohydride is added. The mixture is stirred at room temperature overnight. The ethanol is distilled off and the residue is taken up in 0.5 M hydrochloric acid. The solution is extracted three times with ethyl acetate. The combined organic phases are then dried with sodium sulfate and concentrated. The residue is purified by flash chromatography on silica gel (mobile phase: cyclohexane/ethyl acetate 4:1). 9.3 g of product are obtained.

TLC: R_{f}: 0.28 (cyclohexane/ethyl acetate 1:1).

HPLC (Method 1): Rₜ: 3.40 min.

¹H-NMR (300 MHz, DMSO-d₆, d/ppm): 10.24 (s, broad, 1H), 7.97 (d, 1H), 7.69 (dd, 1H), 6.83 (d, 1H), 5.12 (s, broad, 1H), 4.48 (s, 2H), 4.24 (quart, 2H), 1.29 (t, 3H).

MS (DCI, NH3): m/z=214 (M+NH₄⁺), 410 (2M+NH₄⁺).

### Example II

### [5-Ethoxycarbonyl)-2-hydroxybenzyl](triphenyl)phosphonium bromide

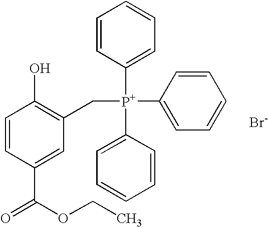

9.3 g (47.40 mmol) of the compound from Example I and 17.08 g (49.77 mmol) of triphenyl-phosphonium bromide are introduced into 400 ml of acetonitrile and stirred at 65° C. overnight. The reaction solution is evaporated to dryness, and the residue is slurried with 100 ml of acetonitrile and filtered off with suction. 23.7 g of product are obtained.

HPLC (Method 1): Rₜ: 4.45 min.

¹H-NMR (300 MHz, DMSO-d₆, d/ppm): 10.72 (s, 1H), 7.92-7.87 (m, 3H), 7.77-7.63 (m, 13H), 7.47 (dd, 1H), 6.82 (d, 1H), 4.98 (d, 2H), 4.13 (quart, 2H), 1.21 (t, 3H).

MS (ESI): m/z=441 (M⁺).

### Example III

### Ethyl 3-[2-(allyloxy)-2-oxoethyl]-4-hydroxybenzoate

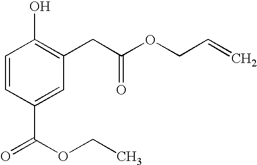

23.7 g (45.46 mmol) of the compound from Example II and 19 ml (136.4 mmol) of triethylamine are introduced into 240 ml of dichloromethane. Then 5.48 g (45.5 mmol) of allyl formate are slowly added dropwise. The mixture is stirred at room temperature overnight. The reaction mixture is poured into 1 l of 10% strength sodium bicarbonate solution, and the aqueous phase is shaken with ethyl acetate. The organic phase is dried over sodium sulfate, absorbed on silica gel and then eluted with cyclohexane/ethyl acetate 4:1. The collected fractions afford after concentration 6.05 g of product.

TLC: R_{f}: 0.45 (cyclohexane/ethyl acetate 1:1).

HPLC (Method 1): Rₜ: 4.22 min.

¹H-NMR (300 MHz, DMSO-d₆, d/ppm): 10.48 (s, broad, 1H), 7.79 (d, 1H), 7.73 (dd, 1H), 6.89 (d, 1H), 5.96-5.83 (m, 1H), 5.30-5.17 (m, 2H), 4.57-4.53 (m, 2H), 4.25 (quart, 2H) 3.68 (s, 2H), 1.29 (t, 3H).

MS (DCI, NH₃): m/z=282 (M+NH₄⁺), 546 (2M+NH₄⁺).

### Example IV

### 3-{4-[(4-Isopropoxybenzyl)oxy]phenyl}propyl methanesulfonate

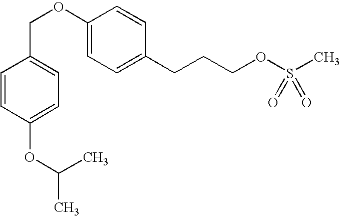

Under an argon atmosphere, 2.0 g (6.66 mmol) of 3-{4[(4-isopropoxybenzyl)oxy]phenyl}propan-1-ol (Example XXI) and 1.1 ml of triethylamine are dissolved in 20 ml of dichloromethane. At 0° C., 0.54 ml (6.99 mmol) of methanesulfonyl chloride is added dropwise. The reaction mixture is slowly brought to room temperature and then stirred for 1 hour. The mixture is then quenched with 10 ml of water, the phases are separated, and the aqueous phase is extracted twice more with dichloromethane. The combined organic phases are washed with saturated sodium chloride solution, dried over sodium sulfate, filtered and concentrated. 2.4 g of product are obtained.

HPLC (Method 2): Rₜ: 5.24 min.

¹-NMR (400 MHz, DMSO-d₆, d/ppm): 7.32 (d, 2H), 7.13 (d, 2H), 6.91 (d, 2H), 6.90 (d, 2H), 4.95 (s, 2H), 4.60 (sep, 1H), 4.18 (t, 2H), 3.16 (s, 3H), 2.60 (t, 2H), 1.92 (m, 2H).

MS (DCI, NH₃): m/z=396 (M+NH₄⁺), 774 (2M+NH₄⁺).

### Example V

### Ethyl 3-[2-(allyloxy)-2-oxoethyl]-4-(3-{4-[(4-isopropoxybenzyl)oxy]phenyl}propoxy)benzoate

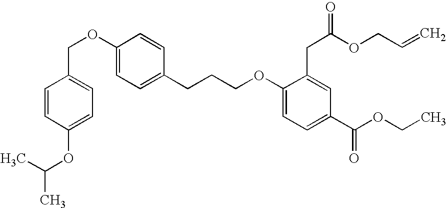

Under an argon atmosphere, 1.52 g (5.76 mmol) of the compound from Example III and 2.40 g (6.34 mmol) of the compound from Example IV and 2.25 g (6.92 mmol) of cesium carbonate are dissolved in 15 ml of N,N-dimethylformamide and stirred at a bath temperature of 60° C. for 4 hours. The reaction solution is mixed with 0.01 M hydrochloric acid and extracted with ethyl acetate. After phase separation, the aqueous phase is extracted once more with ethyl acetate. The combined organic phases are extracted twice with water. The organic phase is dried over sodium sulfate, filtered and concentrated. The residue is purified by flash chromatography (mobile phase: cyclohexane-ethyl acetate 4:1). The collected fractions afford 2.83 g of product.

TLC: R_{f}: 0.64 (cyclohexane/ethyl acetate 1:1).

HPLC (Method 3): Rₜ: 6.00 min.

¹H-NMR (200 MHz, DMSO-d₆, d/ppm): 7.90-7.84 (2H), 7.32 (d, 2H), 7.12-7.02 (3H), 6.93-6.88 (4H), 5.88 (m, 1H), 5.27-5.11 (m, 2H), 4.94 (s, 2H), 4.67-4.52 (3H), 4.28 (quart, 2H), 4.00 (t, 2H), 3.73 (s, 2H), 2.63 (t, 2H), 2.01-1.88 (m, 2H), 1.30 (t, 3H), 1.24 (d, 6H).

MS (ESI): m/z=564 (M+NH₄⁺).

### Example VI

### [5-(Ethoxycarbonyl)-2-(3-{4[(4-isopropoxybenzyl)oxy]phenyl}propoxy)phenyl]ethanoic acid

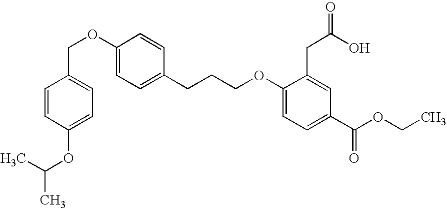

2.8 g (5.12 mmol) of the compound from Example V are dissolved with 0.18 g (0.15 mmol) of tetrakis(triphenylphosphine)palladium(0) in 60 ml of tetrahydrofuran. Subsequently, 6.7 ml (76.83 mmol) of morpholine are then added dropwise and stirred under reflux for 2 hours. The reaction mixture is evaporated to dryness. Purification by preparative RP-HPLC results in 1.99 g of product.

HPLC (Method 2): Rₜ: 5.54 min.

¹H-NMR (300 MHz, DMSO-d₆, d/ppm): 12.23 (s broad, 1H), 7.84 (dd, 1H), 7.81 (d, 1H), 7.32 (d, 2H), 7.12 (d, 2H), 7.02 (d, 1H), 6.89 (d, 2H), 6.88 (d, 2H), 4.93 (s, 2H), 4.59 (sep, 1H), 4.28 (quart, 2H), 4.00 (t, 2H), 3.60 (s, 2H), 2.68 (t, 2H), 1.97 (pseudo-quint, 2H), 1.30 (t, 3H), 1.23 (d, 6H).

MS (ESI): m/z=524 (M+NH₄⁺).

### Example VII

### Methyl (1R,3S)-3-aminocyclopentanecarboxylate hydrochloride

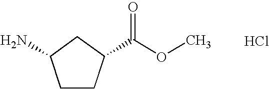

1.0 g (7.74 mmol) of (1R,3S)-3-aminocyclopentanecarboxylic acid is dissolved in 5 ml of methanol, and 2.16 ml (17.03 mmol) of chlorotrimethylsilane are added. The reaction mixture is stirred under reflux overnight. The mixture is then concentrated and dried under high vacuum. 1.16 g of product are obtained.

¹H-NMR (300 MHz, DMSO-d₆, d/ppm): 8.21 (s broad, 3H), 3.62 (s, 3H), 3.47 (m, 1H), 2.86 (m, 1H), 2.25 (m, 1H), 2.01-1.61 (m, 5H).

MS (DCI, NH₃): m/z=144 (M+H⁺), 287 (2M+H⁺).

### Example VIII

### 1-(3-Bromopropyl)-4-(4-phenoxybutoxy)benzene

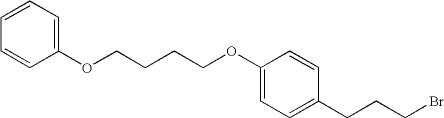

10.0 g (27.5 mmol) of 3-[4-(4-phenoxybutoxy)phenyl]-1-propanol (Example XIX) are dissolved in 50 ml of tetrahydrofuran, and 10.5 g of solid triphenylphosphine are added. 13.2 g of solid tetrabromomethane are added to the solution. After about 5 minutes, the mixture starts to become cloudy. The reaction is completed after one hour. The precipitate is filtered off, and the filtrate is concentrated. The crude product is purified by suction filtration on silica gel with cyclohexane/ethyl acetate 15:1 as mobile phase. 8.5 g of product are obtained.

HPLC (Method 1): Rₜ: 6.01 min.

¹H-NMR (400 MHz, DMSO-d₆, δ/ppm): 7.29-7.24 (m, 2H), 7.12 (d, 2H), 6.94-6.89 (m, 3H), 6.86 (d, 2H), 4.00 (m, 4H), 3.48 (t, 2H), 2.63 (t, 2H), 2.05 (m, 2H), 1.84 (m, 4).

MS (DCI, NH₃): m/z=380/382 (M+NH₄⁺).

### Example IX

### Ethyl 3-[2-(allyloxy)-2-oxoethyl]-4-{3-[4-(4-phenoxybutoxy)phenyl]propoxy}benzoate

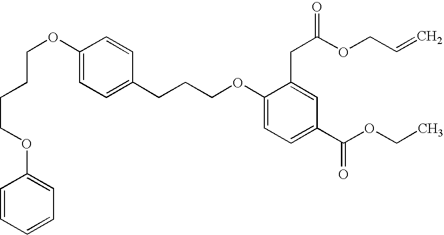

1.5 g (5.68 mmol) of the compound from Example III, 2.06 g (5.68 mmol) of the compound from Example VIII and 1.18 g (8.51 mmol) of potassium carbonate are dissolved in 20 ml of N,N-dimethylformamide and stirred at 65° C. for 1.5 hours. The mixture is brought to room temperature and mixed with about 100 ml of 0.25 M hydrochloric acid, extracted twice with ethyl acetate and washed with saturated sodium chloride solution. After drying, the solvent is stripped off and the residue is purified by flash chromatography (mobile phase: cyclohexane/ethyl acetate 5:1). 2.87 g of product are obtained.

LC-MS (Method 5): Rₜ: 3.23 min, MS (EI+): m/z=547 (M+H⁺).

### Example X

### Ethyl 3-[2-(allyloxy)-2-oxoethyl]-4-{3-[4-4-phenoxybutoxy)phenyl]propoxy}benzoate

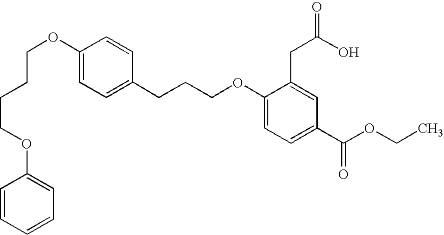

500 mg (0.91 mmol) of the compound from Example IX and 31.7 mg (0.03 mmol) of tetrakis(triphenylphosphine)palladium(0) are dissolved in 10 ml of tetrahydrofuran and then 1.2 ml (13.72 mmol) of morpholine are added dropwise. The reaction mixture is stirred under reflux for 2 hours. The solvent is stripped off and the residue is purified by preparative RP-HPLC. 410 mg of product are obtained.

LC-MS (Method 6): Rₜ: 3.55 min; MS (ESI+): m/z=507 (M+H⁺).

¹H-NMR (200 MHz, DMSO-d₆, d/ppm): 12.29 (s, 1H), 7.80-7.89 (2H), 7.21-7.33 (2H), 6.77-7.17 (8H), 4.28 (quart, 2H), 3.92-4.07 (6H), 3.61 (s, 2H), 2.68 (t, 2H), 1.96 (t, 2H), 1.79-1.90 (4H), 1.30 (t, 3H).

### Example XI

### Ethyl 3-(2-chloro-2-oxoethyl)-4-{3-[4-(4-phenoxybutoxy)phenyl]propoxy}benzoate

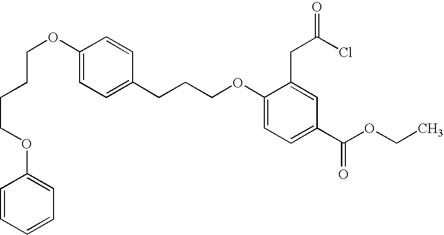

Under an argon atmosphere, 650 mg (1.28 mmol) of the compound from Example X are introduced into 15 ml of tetrahydrofuran. At room temperature, 0.94 ml (12.83 mmol) of thionyl chloride is added dropwise, and the mixture is stirred at this temperature overnight. The mixture is concentrated in a rotary evaporator. 900 mg of crude product are obtained and are reacted without further purification in Example 10.

### Example XII

### 1-(3-Bromopropyl)-4-[4-(cyclohexyloxy)butoxy]benzene

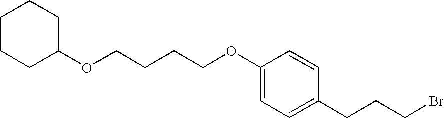

10.0 g of 3-{4-[4-cyclohexyloxy)butoxy]phenyl}propan-1-ol-(Example XXIII) are dissolved in 50 ml of tetrahydrofuran, and 10.5 g of solid triphenylphophine are added. 13.2 g of solid tetrabromomethane are added to the solution. After about 5 minutes, the mixture starts to become cloudy. The reaction is complete after one hour. The precipitate is filtered off and the filtrate is concentrated. The crude product is purified by suction filtration on silica gel with cyclohexane/ethyl acetate 15:1 as mobile phase. 8.5 g of product are obtained.

HPLC (Method 1): Rₜ: 6.01 min.

¹H-NMR (400 MHz, DMSO-d₆, d/ppm): 7.29-7.24 (m, 2H), 7.12 (d, 2H), 6.94-6.89 (m, 3H), 6.86 (d, 2H), 4.00 (m, 4H), 3.48 (t, 2H), 2.63 (t, 2H), 2.05 (m, 2H), 1.84 (m, 4).

MS (DCI, NH₃): m/z=380/382 (M+NH₄⁺).

### Example XIII

### Ethyl 3-[2-(allyloxy)-2-oxoethyl]-4-(3-{4-[4-(cyclohexyloxy)butoxy]phenyl}propoxy)benzoate

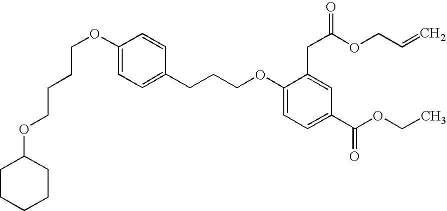

Under an argon atmosphere, 2.0 g (7.57 mmol) of the compound from Example III and 3.07 g (8.32 mmol) of the compound from Example XII are mixed with 2.96 g (9.08 mmol) of cesium carbonate and dissolved in 15.00 ml of N,N-dimethylformamide. The reaction mixture is stirred at a bath temperature of 60° C. for 4 hours. The reaction solution is mixed with 0.01 M hydrochloric acid and extracted twice with ethyl acetate. The combined organic phases are washed twice with water, dried over sodium sulfate, filtered and concentrated. The residue is purified by flash chromatography (mobile phase: cyclohexane/ethyl acetate 4:1). 3.38 g of product are obtained.

TLC: R_{f}: 0.64 (cyclohexane/ethyl acetate 1:1).

HPLC (Method 2): Rₜ: 6.46 min.

¹H-NMR (300 MHz, DMSO-d₆, d/ppm): 7.88 (d, 1H), 7.85 (s, 1H), 7.09 (d, 2H), 7.04 (d, 1H), 6.82 (d, 2H), 5.88 (m, 1H), 5.24-5.12 (m, 2H), 4.53 (d, 2H), 4.28 (quart, 2H), 3.99 (t, 2H), 3.92 (t, 2H), 3.73 (s, 2H), 3.42 (t, 2H), 3.21 (m, 1H), 2.63 (t, 2H), 1.93 (pseudo-quint, 2H), 1.79-1.55 (8H), 1.47 (m, 1H), 1.29 (t, 3H), 1.19 (5H).

MS (ESI): m/z=553 (M+H⁺), 570 (M+NH₄⁺).

### Example XIV

### [2-(3-{4-[4-(Cyclohexyloxy)butoxy]phenyl}propoxy)-5-(ethoxycarbonyl)phenyl]ethanoic acid

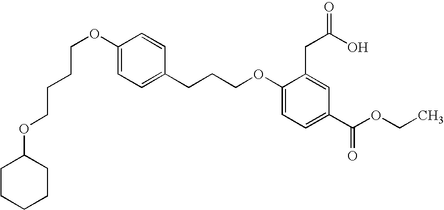

3.34 g (6.04 mmol) of the compound from Example XIII and 0.21 g (0.18 mmol) of tetrakis(triphenylphosphine)palladium(0) are dissolved in 60 ml of tetrahydrofuran, and then 7.91 ml (90.65 mmol) of morpholine are added dropwise. The reaction mixture is stirred under reflux for 2 hours. The mixture is then evaporated to dryness. The residue is purified by preparative RP-HPLC. 2.38 g of product are obtained.

HPLC (Method 2): Rₜ: 5.71 min.

¹H-NMR (300 MHz, DMSO-d₆, d/ppm): 12.23 (s broad, 1H), 7.83 (d, 1H), 7.81 (s, 1H), 7.10 (d, 2H), 7.02 (d, 1H), 6.81 (d, 2H), 4.28 (quart, 2H), 3.99 (t, 2H), 3.92 (t, 2H), 3.60 (s, 2H), 3.41 (t, 2H), 3.20 (m, 1H), 2.68 (t, 2H), 1.97 (pseudo-quint, 2H), 1.82-1.53 (8H), 1.46 (m, 1H), 1.30 (t, 3H), 1.19 (5H).

MS (ESI): m/z=513 (M+H⁺), 530 (M+NH₄⁺).

### Example XV

### Methyl azetidine-3-carboxylate hydrochloride

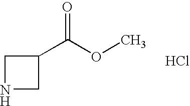

190 mg (1.88 mmol) of 3-azetidinecarboxylic acid are introduced into 7 ml of methanol and cooled to 0° C. 0.15 ml (2.07 mmol) of thionyl chloride is added dropwise. The reaction mixture is slowly brought to room temperature and stirred at this temperature overnight. The methanol is removed in a rotary evaporator and the residue is dried under high vacuum. 271 mg of product are obtained and are reacted as in Example 20 without further purification.

¹H-NMR (300 MHz, DMSO-d₆, d/ppm): 9.44 (s, broad, 1H); 9.16 (s, broad, 1H), 4.17-3.94 (m, 4H), 3.70 (s, 3H).

MS (DCI, NH₃): m/z=116 (M+H⁺), 133 (M+NH₄⁺), 231 (2M+H⁺).

### Example XVI

### Ethyl 3-[2-(allyloxy)-2-oxoethyl]-4-{3-[4-(3-cyclohexylpropoxy)phenyl]propoxy}benzoate

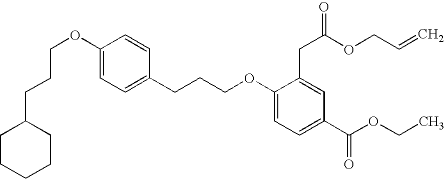

Under an argon atmosphere, 2.0 g (7.57 mmol) of the compound from Example III and 2.82 g (8.32 mmol) of 1-(3-bromopropyl)-4-(3-cyclohexylpropoxy)benzene (Example XXVI) are introduced into 15 ml of N,N-dimethylformamide and stirred with 2.96 g (9.08 mmol) of cesium carbonate at 60° C. for 4 hours. After cooling to room temperature, the reaction solution is mixed with 0.01 M hydrochloric acid and extracted several times with ethyl acetate. The combined organic phases are washed twice with water, dried over sodium sulfate and concentrated in a rotary evaporator. The residue is purified by flash chromatography on silica gel (mobile phase: cyclohexane/ethyl acetate 4:1). 3.16 g of product are obtained.

TLC: R_{f}: 0.64 (cyclohexane/ethyl acetate 1:1).

HPLC (Method 2): Rₜ: 7.22 min.

¹H-NMR (300 MHz, DMSO-d₆, d/ppm): 7.88 (d, 1H), 7.87 (s, 1H), 7.08 (d, 2H), 7.04 (d, 1H), 6.81 (d, 2H), 5.88 (m, 1H), 5.25-5.13 (m, 2H), 4.53 (d, 2H), 4.28 (quart, 2H), 4.00 (t, 2H), 3.89 (t, 2H), 3.72 (s, 2H), 2.63 (t, 2H), 1.94 (pseudo-quint, 2H), 1.72-1.58 (7H), 1.32-1.12 (9H), 0.88 (m, 2H).

MS (ESI): m/z=523 (M+H⁺), 540 (M+NH₄⁺).

### Example XVII

### [2-{3-[4-(3-Cyclohexylpropoxy)phenyl]propoxy}-5-(ethoxycarbonyl)phenyl]ethanoic acid

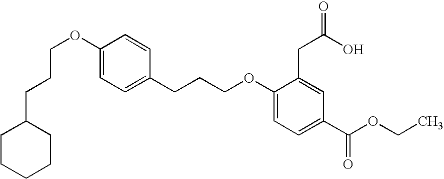

3.10 g (5.93 mmol) of the compound from Example XVI and 0.21 g (0.18 mmol) of tetrakis(triphenylphosphine)palladium(0) are dissolved in 60 ml of tetrahydrofuran and subsequently 7.76 ml (88.96 mmol) of morpholine are added dropwise. The reaction mixture is stirred under reflux for 2 hours. The mixture is then evaporated to dryness and the residue is purified by preparative RP-HPLC. 1.56 g of product are obtained.

HPLC (Method 2): Rₜ: 6.13 min.

¹H-NMR (300 MHz, DMSO-d₆, d/ppm): 7.84 (d, 1H), 7.81 (s, 1H), 7.10 (d, 2H), 7.01 (d, 1H), 6.81 (d, 2H), 4.28 (quart, 2H), 4.00 (t, 2H), 3.88 (t, 2H), 3.60 (s, 2H), 2.67 (t, 2H), 1.96 (pseudo-quint, 2H), 1.71-1.60 (7H), 1.32-1.10 (9H), 0.88 (m, 2H).

MS (ESI): m/z=500 (M+NH₄⁺).

### Example XVIII

### Methyl 3-[4-(4-phenoxybutoxy)phenyl]propanoate

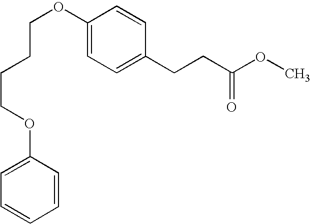

A solution of 10.0 g of methyl 3-(4-hydroxyphenyl)propanoate and 12.7 g of 4-phenoxy-1-butyl bromide in 100 ml of acetonitrile is mixed with 11.5 g of potassium carbonate and heated to reflux for 15 hours. The solvent is then removed in a rotary evaporator, and the residue is taken up in ethyl acetate and water. After phase separation, the aqueous phase is extracted with ethyl acetate. The combined organic phases are washed with saturated sodium chloride solution and dried over anhydrous sodium sulfate. The crude product after evaporation is purified by suction filtration through silica gel with cyclohexane/ethyl acetate 19:1. 12.6 g of product are obtained.

TLC: R_{f}: 0.36 (cyclohexane/ethyl acetate 1:1).

HPLC (method 1): Rₜ: 5.41 min.

¹H-NMR (200 MHz, DMSO-d₆, d/ppm): 7.32-7.23 (m, 2H), 7.11 (d, 2H), 6.96-6.82 (m, 5H), 3.98 (m, 4H), 3.57 (s, 3H), 2.77 (t, 3H), 2.57 (t, 2H), 1.85 (m, 4H).

MS (ESI+): m/z=329 (M+H⁺), 351 (M+Na⁺).

### Example XIX

### 3-[4-(4-phenoxybutoxy)phenyl]-1-propanol

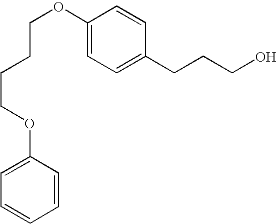

20.1 ml of a 1 molar solution of lithium aluminium hydride in tetrahydrofuran is introduced. While stirring, a solution of 12.0 g of methyl 3-[4-(4-phenoxybutoxy)phenyl]propanoate in 40 ml of THF is added dropwise in such a way that the mixture just starts to boil. The mixture is stirred at room temperature for 30 minutes. 1 ml of methanol is cautiously added to the suspension in order to hydrolyze excess lithium aluminium hydride. The mixture is then added to 1 molar hydrochloric acid and extracted with ethyl acetate. The organic phase is separated off, washed with saturated sodium chloride solution and dried over anhydrous sodium sulfate. Filtration and evaporation result in 10.9 g of product.

HPLC (method 1): Rₜ: 4.94 min.

¹H-NMR (300 MHz, DMSO-d₆, d/ppm): 7.30-7.24 (m, 2H), 7.08 (d, 2H), 6.95-6.88 (m, 3H), 6.83 (d, 2H), 4.39 (t, 1H), 4.01 (m, 4H), 3.38 (quart, 2H), 2.53 (t, 2H), 1.83 (m, 4H), 1.67 (m, 2H).

MS (ESI+): m/z=301 (M+H⁺), 323 (M+Na⁺).

### Example XX

### Methyl 3-{4-[(4-isopropoxybenzyl)oxy]phenyl}propanoate

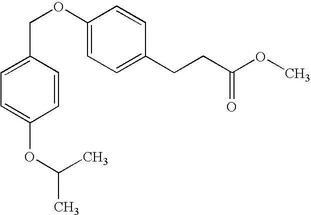

A solution of 11.0 g of 4-isopropoxybenzyl chloride and 10.7 g of methyl 3-(4-hydroxyphenyl)-propanoate in 120 ml of butyronitrile is mixed with 12.4 g of potassium carbonate and heated to reflux for 15 hours. The solvent is then distilled out in a rotary evaporator. The residue is taken up in ethyl acetate and washed successively with water and saturated sodium chloride solution. After drying over sodium sulfate, the organic phase is evaporated and the product is purified by suction filtration through silica with cyclohexane/ethyl acetate 9:1. 9.4 g of product are obtained.

HPLC (Method 1): Rₜ: 5.33 min.

¹H-NMR (300 MHz, DMSO-d₆, d/ppm): 7.32 (d, 2H), 7.11 (d, 2H), 6.90 (d, 2H), 6.89 (d, 2H), 4.94 (s, 2H), 4.60 (sep, 1H), 3.57 (s, 3H), 2.78 (t, 2H), 2.57 (t, 2H), 1.27 (d, 6H).

MS (DCI, NH₃): m/z=346.2 (M+NH₄⁺).

### Example XXI

### 3-{4-[(4-Isopropoxybenzyl)oxy]phenyl}propan-1-ol

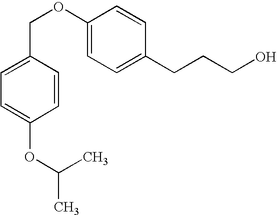

3-{4-[(4-Isopropoxybenzyl)oxy]phenyl}propan-1-ol is obtained by reduction of methyl 3-{4-[(4-isopropoxybenzyl)oxy]phenyl}propanoate with lithium aluminium hydride in analogy to the process described in Example XIX.

TLC: R_{f}: 0.48 (cyclohexane/ethyl acetate 1:1).

HPLC (Method 1): Rₜ: 4.87 min.

¹H-NMR (300 MHz, CDCl₃, d/ppm): 7.32 (d, 2H), 7.11 (d, 2H), 6.90 (d, 2H), 6.89 (d, 2H), 4.93 (s, 2H), 4.54 (sep, 1H), 3.68 (t broad, 2H), 2.64 (t, 2H), 1.93-1.79 (m, 2H), 1.33 (d, 6H), 1.26 (s broad, 1H).

MS (DCI, NH₃): m/z=318 (M+NH₄⁺).

### Example XXII

### Methyl 3-{4-[4-(cyclohexyloxy)butoxy]phenyl}propanoate

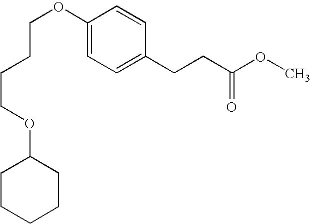

Methyl 3-{4-[4-(cyclohexyloxy)butoxy]phenyl}propanoate is obtained by alkylation of methyl 3-(4-hydroxyphenyl)propanoate with (4-bromobutoxy)cyclohexane in analogy to the process described in Example XVIII.

¹H-NMR (200 MHz, CDCl₃, d/ppm): 7.09 (d, 2H), 6.82 (d, 2H), 3.94 (t, 2H), 3.67 (s, 3H), 3.49 (t, 2H), 3.30-3.15 (m, 1H), 2.88 (t, 2H), 2.59 (t, 2H), 1.94-1.17 (m, 14H).

LC-MS (Method 7): Rₜ: 3.01 min, m/z=335 (M+H⁺).

### Example XXIII

### 3-{4-[4-(Cyclohexyloxy)butoxy]phenyl}propan-1-ol

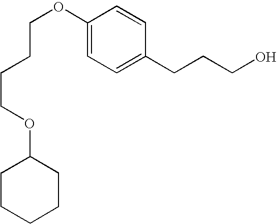

3-{4-[4-(Cyclohexyloxy)butoxy]phenyl}propan-1-ol is obtained by reduction of methyl 3-{4-[4-(cyclohexyloxy)butoxy]phenyl}propanoate with lithium aluminium hydride in analogy to the process described in Example XIX.

¹H-NMR (400 MHz, CDCl₃, d/ppm): 7.10 (d, 2H), 6.82 (d, 2H), 3.95 (t, 2H), 3.67 (t, 2H), 3.49 (t, 2H), 3.23-3.19 (m, 1H), 2.63 (t, 2H), 1.92-1.20 (16H).

LC-MS (Method 7): Rₜ: 2.67 min, m/z=307 (M+H⁺).

### Example XXIV

### Methyl 3-[4-(3-cyclohexylpropoxy)phenyl]propanoate

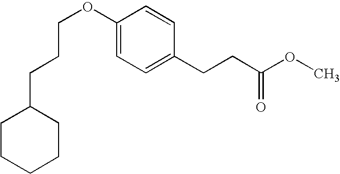

A solution of 32.21 g of methyl 3-(4-hydroxyphenyl)propanoate and 44.0 g of 1-bromo-3-cyclohexylpropane in 100 ml of anhydrous DMF is mixed with 69.9 g of cesium carbonate and stirred at a temperature of 50° C. for 6 hours. The reaction mixture is then poured into about 800 ml of water, mixed with 12.3 ml of glacial acetic acid and extracted three times with ethyl acetate. The combined organic extracts are washed successively with water and saturated sodium chloride solution. After drying over anhydrous sodium sulfate, the solvent is removed in a rotary evaporator. The product is purified by suction filtration on silica gel with cyclohexane/ethyl acetate 20:1 as mobile phase. 51.52 g are obtained.

TLC: R_{f}: 0.47 (cyclohexane/ethyl acetate 1:1).

HPLC (Method 2): Rₜ: 6.09 min.

¹H-NMR (300 MHz, DMSO-d₆, d/ppm): 7.10 (d, 2H), 6.81 (d, 2H), 3.88 (t, 2H), 3.57 (s, 3H), 2.77 (t, 2H), 2.57 (t, 2H), 1.72-1.64 (m, 7H), 1.37-1.09 (m, 6H), 0.93-0.81 (m, 2H).

MS (DCI, NH₃): m/z=321.9 (M+NH₄⁺).

### Example XXV

### 3-[4-(3-Cyclohexylpropoxy)phenyl]propan-1-ol

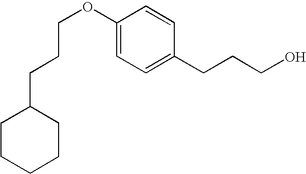

3-[4-(3-Cyclohexylpropoxy)phenyl]propan-1-ol is obtained in analogy to the process described in Example XIX by reduction of methyl 3-[4-(3-cyclohexylpropoxy)phenyl]propanoate with lithium aluminium hydride.

TLC: R_{f}: 0.30 (cyclohexane/ethyl acetate 1:1).

HPLC (method 1): Rₜ: 5.60 min.

¹H-NMR (300 MHz, DMSO-d₆, d/ppm): 7.08 (d, 2H), 6.80 (d, 2H), 4.38 (t, 1H), 3.88 (t, 2H), 3.39 (quart., 2H), 2.51 (t, 2H, concealed by the DMSO signal), 1.71-1.61 (m, 9H), 1.32-1.12 (m, 6H), 0.93-0.81 (m, 2H).

MS (DCI, NH₃): m/z=394.1 (M+NH₄⁺).

### Example XXVI

### 1-(3-Bromopropyl)-4-(3-cyclohexylpropoxy)benzene

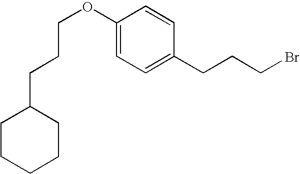

1-(3-Bromopropyl)-4-(3-cyclohexylpropoxy)benzene is obtained in analogy to the process described in Example VIII from 3-[4-(3-cyclohexylpropoxy)phenyl]propan-1-ol by bromination with triphenylphosphine and tetrabromomethane.

TLC: R_{f}: 0.69 (cyclohexane/ethyl acetate 1:1).

HPLC (method 3): Rₜ: 7.27 min.

¹H-NMR (300 MHz, DMSO-d₆, d/ppm): 7.10 (d, 2H), 6.82 (d, 2H), 3.89 (t, 2H), 3.48 (t, 2H), 2.62 (t, 2H), 2.03 (quint., 2H), 1.73-1.60 (m, 7H), 1.30-1.12 (m, 6H), 0.93-0.81 (m, 2H).

MS (DCI, NH₃): m/z=338 and 340 (M⁺), 356 and 358 (M+NH₄⁺).

### Example XXVII

### Methyl cis-3-hydroxycyclohexanecarboxylate

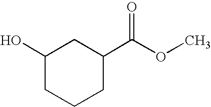

A total of 2.51 g of solid sodium borohydride is added in portions to a solution of 20.7 g of methyl 3-oxocyclohexanecarboxylate in 520 ml of methanol at a temperature of −78° C. After 2.5 hours at −78° C., 5 ml of water are added, and the reaction mixture is allowed to reach room temperature. The solvent is then removed in a rotary evaporator. Extraction is carried out with ethyl acetate from water. Drying of the organic phase over anhydrous sodium sulfate, filtration and removal of the ethyl acetate results in 19.0 g of an oil. According to N, the product consists of a mixture of two diastereomers in the ratio 94:6. In analogy to similar reductions described in the literature for cyclohexanones substituted in position 3 with sodium borohydride it is concluded that the main product must be the cis isomer.

¹H-NMR (300 MHz, DMSO d₆, d/ppm, main isomer): 4.60 (d, 1H), 3.58 (s, 3H), 3.43-3.32 (m, 1H), 2.32 (tt, 1H), 2.03-1.97 (m, 1H), 1.82-1.67 (m, 3H), 1.31-0.96 (m, 4H).

MS (DCI, NH₃): m/z=159 (M+H⁺), 176 (M+NH₄⁺).

### Example XXVIII

### Methyl cis-3-{[(4-methylphenyl)sulfonyl]oxy}cyclohexanecarboxylate

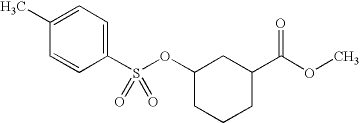

32 g of para-toluenesulfonyl chloride are added to a solution of 18.97 g of methyl cis-3-hydroxycyclohexanecarboxylate and 97 ml of pyridine in 410 ml of anhydrous dichloromethane at a temperature of 0° C. The cooling bath is removed, and the reaction mixture is stirred at room temperature overnight. The solvent and excess pyridine are then removed in a rotary evaporator. The residue obtained is taken up in ethyl acetate. Insolubles are filtered off and the filtrate is concentrated. The solid residue is stirred a total of six times with about 500 ml of petroleum ether each time. This results in 29.7 g of a crystalline solid.

HPLC (Method 1): Rₜ: 4.67 min.

¹H-NMR (300 MHz, DMSO d₆, d/ppm): 7.79 (d, 2H), 7.48 (d, 2H), 4.53-4.44 (m, 1H), 3.57 (s, 3H), 2.42 (s, 3H), 2.07-1.99 (m, 1H), 1.78-1.69 (m, 3H), 1.49 (quart., 1H), 1.41-1.13 (m, 3H).

MS (DCI, NH₃): m/z=330 (M+NH₄⁺).

### Example XXIX

### Methyl trans-3-azidocyclohexanecarboxylate

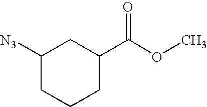

A solution of 29.6 g of methyl cis-3-{[(4-methylphenyl)sulfonyl]oxy}cyclohexanecarboxylate in 660 ml of DMF is mixed with 6.49 g of sodium azide and stirred at 80° C. for 15 hours. 1000 ml of water are added at room temperature, and the product is extracted three times with about 300 ml of diethyl ether each time. The organic extract is washed with saturated sodium chloride solution and dried over anhydrous sodium sulfate. Filtration and concentration in a rotary evaporator at room temperature result in 16.8 g of an oil. Assignment of the product to the transisomer is derived from the experience that reactions of this type take place with inversion of the stereochemistry at the reaction site.

¹H-NMR (200 MHz, DMSO-d₆, d/ppm): 3.94 (quint., 1H), 3.60 (s, 3H), 2.68-2.56 (m, 1H), 1.82-1.72 (m, 3H), 1.62-1.43 (m, 5H).

MS (DCI, NH₃): m/z=201 (M+NH₄⁺), 218 (M+N₂H₇⁺).

### Example XXX

### Methyl trans-3-aminocyclohexanecarboxylate hydrochloride

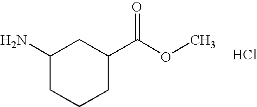

Method A:

A solution of 16.45 g of methyl trans-3-azidocylohexanecarboxylate in 502 ml of methanol is first mixed with 1.68 g of 10% palladium on carbon and 17.29 g of ammonium formate and then heated to reflux for 1 hour. This is followed by filtrations through a little Tonsil and concentration in a rotary evaporator. The residue is dissolved in a little ethyl acetate and mixed with 30 ml of 4 molar hydrogen chloride in dioxane. After 20 minutes, the mixture is evaporated to dryness. 16.3 g of a solid are obtained.

¹H-NMR (400 MHz, DMSO-d₆, d/ppm): 8.23 (s broad, 3H), 3.61 (s, 3H), 3.07-3.00 (m, 1H), 2.51-2.42 (m, 1H), 2.17 (d broad, 1H), 1.93-1.74 (m, 3H), 1.42-1.11 (m, 4H).

MS (DCI, NH₃): m/z=158 (M+H⁺).

Method B:

Methyl trans-3-aminocyclohexanecarboxylate hydrochloride can also be obtained by the process described for Method A from methyl trans-3-azidocyclohex-4-enecarboxylate. In this case merely the amount of ammonium formate employed is doubled.

### Example XXXI

### trans-3-Azidocyclohex-4-enecarboxylic acid

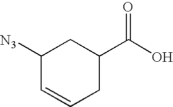

A solution of 18.4 g of racemic cis-6-oxabicyclo[3.2.1]oct-3-en-7-one in 175 ml of THF is mixed with a solution of 10.6 of sodium azide in 70 ml of water. The reaction mixture is then heated to reflux for 15 hours. The THF is subsequently stripped off in a rotary evaporator at a bath temperature of 30° C. The remaining aqueous phase is mixed with 165 ml of 2 molar sodium hydroxide solution and extracted twice with 110 ml of toluene each time and once with diethyl ether. The aqueous phase is then acidified with concentrated hydrochloric acid at about 10° C. The product is extracted with dichloromethane. The organic extract is dried over anhydrous sodium sulfate, filtered and concentrated. 19.7 g of an oil are obtained and become solid on storage in a refrigerator. Assignment of the product to the transisomer is derived from the experience that reactions of this type take place with inversion of the stereochemistry at the reaction site.

HPLC (Method 1): Rₜ: 3.62 min.

¹H-NMR (300 MHz, DMSO-d₆, d/ppm): 12.38 (s, broad, 1H), 6.09-6.03 (m, 1H), 5.82-5.77 (m, 1H), 4.14 (pseudo-d, 1H), 2.59-2.48 (m, 1H, partly concealed by DMSO signal), 2.37-2.28 (m, 1H), 2.18-2.07 (m, 1H), 2.01-1.96 (m, 1H), 1.82-1.73 (m, 1H).

MS (ESI−): m/z=166 (M−H⁻), 333 (2M−H⁻).

### Example XXXII

### Methyl trans-3-azidocyclohex-4-enecarboxylate

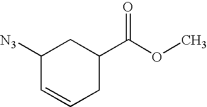

32.6 ml of trimethylsilyl chloride are added dropwise to a solution of 19.5 g of trans-3-azidocyclohex-4-enecarboxylic acid in 1.5 l of anhydrous methanol at 0° C. After 1 hour at 0° C., the mixture is stirred at room temperature for a further hour. It is then evaporated to dryness. 19.1 g of an oil are obtained.

TLC (cyclohexane/ethyl acetate 1:1): R_{f}: 0.75.

HPLC (Method 1): Rₜ: 4.24 min.

¹H-NMR (300 MHz, DMSO-d₆, d/ppm): 6.08-6.02 (m, 1H), 5.82-5.77 (m, 1H), 4.17 (pseudo-d, 1H), 3.62 (s, 3H), 2.69-2.59 (m, 1H), 2.38-2.29 (m, 1H), 2.20-2.08 (m, 1H), 2.02-1.94 (m, 1H), 1.85-1.76 (m, 1H).

MS (DCI, NH₃): m/z=199 (M+NH₄⁺).

### Exemplary Embodiments

### Example 1

### Methyl 1-{[5-(ethoxycarbonyl)-2-(3-{4-[(4-isopropoxybenzyl)oxy]phenyl}propoxy)phenyl]-acetyl}piperidine-4-carboxylate

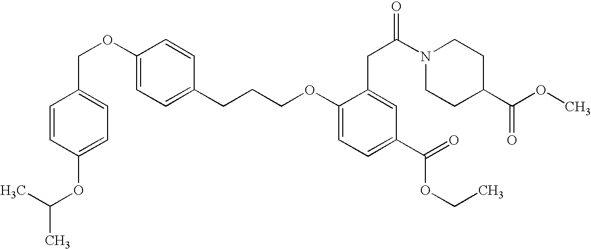

197.6 mg (0.39 mmol) of the compound from Example VI are introduced under an argon atmosphere in 20 ml of dichloromethane, 61.4 mg (0.43 mmol) of methyl isonipectotate are added and then, in the stated sequence, 50 mg (0.39 mmol) of 1-hydroxy-1H-benzotriazole hydrate, 149 mg (0.78 mmol) of N′-(3-dimethylaminopropyl)-N-ethylcarbodiimide hydrochloride and 0.11 ml (0.78 mmol) of triethylamine are added. The reaction mixture is stirred at room temperature overnight. The mixture is evaporated to dryness, taken up in 3 ml of dimethyl sulfoxide and filtered. The filtrate is purified with the aid of preparative RP-HPLC. 186 mg of product are obtained.

TLC: R_{f}: 0.31 (cyclohexane/ethyl acetate 1:1).

HPLC (Method 2): Rₜ: 5.70 min.

MS (DCI, NH₃): m/z=632 (M+H⁺), 654 (M+NH₄⁺).

### Example 2

### 1-{[5-Carboxy-2-(3-{4-[(4-isopropoxybenzyl)oxy]phenyl}propoxy)phenyl]acetyl}piperidine-4-carboxylic acid

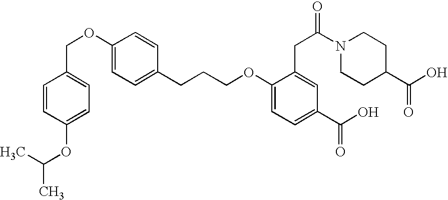

166 mg (0.26 mmol) of the compound from Example 1 are dissolved in 2 ml of tetrahydrofuran and 2 ml of methanol. Then 2 ml of 2 M sodium hydroxide solution are added to the solution and the mixture is stirred at 60° C. for 1 hour. The reaction mixture is freed of volatiles, and 2.1 ml of 2 M hydrochloric acid are added to the aqueous residue. The product then precipitates. It is filtered off with suction and washed with water. Drying under high vacuum results in 101 mg of product.

HPLC (Method 2): Rₜ: 4.87 min.

¹H-NMR (300 MHz, DMSO-d₆, d/ppm): 12.36 (s broad, 2H), 7.80 (dd, 1H), 7.72 (d, 1H), 7.32 (d, 2H), 7.11 (d, 2H), 6.99 (d, 1H), 6.91 (d, 2H), 6.89 (d, 2H), 4.93 (s, 2H), 4.60 (sep, 1H), 4.21 (m, 1H), 3.98 (t, 2H), 3.91 (m, 1H), 3.68 (2H), 3.12 (m, 1H), 2.73 (m, 1H), 2.65 (t, 2H), 2.49 (m, 1H, partly concealed by DMSO signal), 1.97 (pseudo-quint, 2H), 1.81 (m, 2H), 1.51-1.29 (2H), 1.25 (d, 6H).

MS (ESI): m/z=590 (M+H⁺).

### Example 3

### Ethyl-4-(3-{4-[(4-isopropoxybenzyl)oxy]phenyl}propoxy)-3-{2-[(4-methoxy-4-oxobutyl)amino]-2-oxoethyl}benzoate

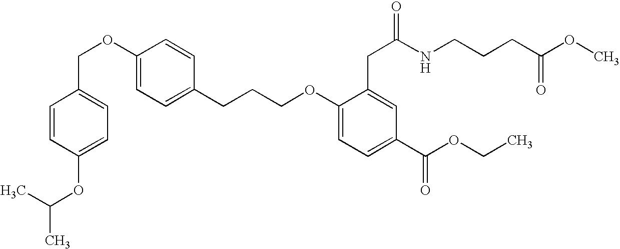

Under an argon atmosphere, 200 mg (0.39 mmol) of the compound from Example VI are introduced into 20 ml of dichloromethane, and 50.8 g (0.43 mmol) of methyl 4-aminobutyrate hydrochloride are added and then, in the stated sequence, 50 mg (0.39 mmol) of 1-hydroxy-1H-benzotriazole hydrate, 151.4 mg (0.79 mmol) of N′-(3-dimethylaminopropyl)-N-ethylcarbodiimide hydrochloride and 0.11 ml (0.79 mmol) of triethylamine are added. The reaction mixture is stirred at room temperature overnight. The mixture is evaporated to dryness, taken up in 3 ml of dimethyl sulfoxide and filtered. Subsequent purification by preparative RP-HPLC affords 151 mg of product.

TLC: R_{f}: 0.25 (cyclohexane/ethyl acetate 1:1).

HPLC (Method 2): Rₜ: 5.49 min.

¹H-NMR (200 MHz, DMSO-d₆, d/ppm): 7.97 (t, 1H), 7.82 (dd, 1H), 7.79 (d, 1H), 7.32 (d, 2H), 7.12 (d, 2H), 7.00 (d, 1H), 6.90 (d, 2H), 6.88 (d, 2H), 4.93 (s, 2H), 4.60 (sep, 1H), 4.27 (quart, 2H), 3.98 (t, 2H), 3.53 (s, 3H), 3.47 (s, 2H), 3.07 (quart, 2H), 2.68 (t, 2H), 2.29 (t, 2H), 1.97 (pseudo-quint, 2H), 1.65 (t, 3H), 1.30 (t, 3H), 1.23 (d, 6H).

MS (ESI): m/z=606 (M+H⁺).

### Example 4

### 3-{2-[(3-Carboxypropyl)amino]-2-oxoethyl}-4-(3-{4-[(4-isopropoxybenzyl)oxy]phenyl}-propoxy)benzoic acid

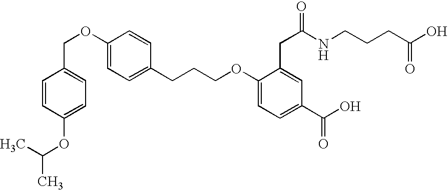

131 mg (0.22 mmol) of the compound from Example 3 are dissolved in 2 ml of tetrahydrofuran and 2 ml of methanol. The mixture is then mixed with 2 ml of 2 M sodium hydroxide solution and stirred at 60° C. for 1 hour. The reaction mixture is freed of volatiles, and 2.1 ml of 2 M hydrochloric acid are added to the aqueous residue. The product precipitates. It is filtered off with suction and washed with water. Drying under the high vacuum results in 47 mg of product.

HPLC (Method 2): Rₜ: 4.76 min.

¹H-NMR (300 MHz, DMSO-d₆, d/ppm): 12.26 (s broad, 2H), 7.91 (t, 1H), 7.80 (dd, 1H), 7.77 (d, 1H), 7.32 (d, 2H), 7.12 (d, 2H), 6.97 (d, 1H), 6.90 (d, 2H), 6.88 (d, 2H), 4.93 (s, 2H), 4.59 (sep, 1H), 3.97 (t, 2H), 3.44 (s, 2H), 3.07 (quart, 2H), 2.68 (t, 2H), 2.20 (t, 2H), 1.97 (pseudo-quint, 2H), 1.62 (quint, 2H), 1.23 (d, 6H).

MS (ESI): m/z=564 (M+H⁺).

### Example 5

### Ethyl-4-(3-{4-[(4-Isopropoxybenzyl)oxy]phenyl}propoxy)-3-(2-{[(1S,3R)-3-(methoxycarbonyl)-cyclopentyl]amino}-2-oxoethyl)benzoate

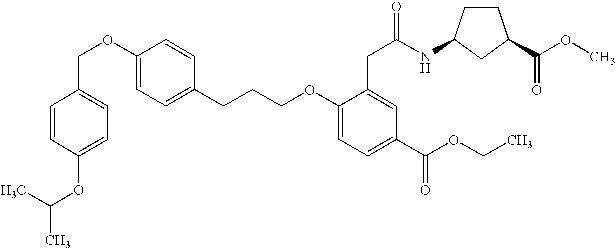

200 mg (0.39 mmol) of the compound from Example VI are introduced into 20 ml of dichloromethane, and 78 mg (0.43 mmol) of the compound from Example VII are added. Then, in the stated sequence, 50 mg (0.39 mmol) of 1-hydroxy-1H-benzotriazole hydrate, 151 mg (0.79 mmol) of N′-(3-dimethylaminopropyl)-N-ethylcarbodiimide hydrochloride and 0.11 ml (0.79 mmol) of triethylamine are added. The reaction mixture is stirred at room temperature overnight. The mixture is evaporated to dryness, taken up in 3 ml of dimethyl sulfoxide and filtered. Subsequent purification by preparative RP-HPLC affords 179 mg of product.

TLC: R_{f}: 0.23 (cyclohexane/ethyl acetate 1:1).

HPLC (Method 2): Rₜ: 5.57 min.

¹H-NMR (200 MHz, DMSO-d₆, d/ppm): 8.03 (d, 1H), 7.82 (dd, 1H), 7.78 (d, 1H), 7.32 (d, 2H), 7.12 (d, 2H), 7.00 (d, 1H), 6.90 (d, 2H), 6.88 (d, 2H), 4.93 (s, 2H), 4.60 (sep, 1H), 4,27 (quart, 2H), 4.09-3.94 (3H), 3.58 (s, 3H), 3.49 (s, 2H), 2.83-2.64 (3H), 2.20-2.06 (m, 1H), 2.01-1.92 (m, 2H), 1.83-1.77 (3H), 1.68-1.41 (2H), 1.29 (t, 3H), 1.24 (d, 6H).

MS (ESI): m/z=632 (M+H⁺).

### Example 6

### 3-(2-{[(1S,3R)-3-Carboxycyclopentyl]amino}-2-oxoethyl)-4-(3-{4-[(4-isopropoxybenzyl)oxy]-phenyl}propoxy)benzoic acid

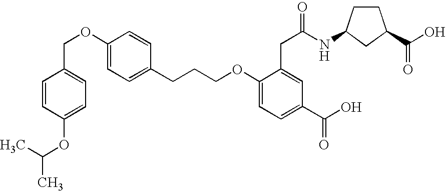

164 mg (0.26 mmol) of the compound from Example 5 are dissolved in 2 ml of tetrahydrofuran and 2 ml of methanol. The mixture is then mixed with 2 ml of 2 M sodium hydroxide solution and stirred at 60° C. for 1 hour. The reaction mixture is then freed of volatiles in a rotary evaporator, and 2.1 ml of 2 M hydrochloric acid are added to the aqueous residue. The product precipitates. It is filtered off with suction and washed with a little water. Drying under high vacuum results in 127 mg of product.

HPLC (Method 2): Rₜ: 4.80 min.

¹H-NMR (300 MHz, DMSO-d₆, d/ppm): 12.27 (s broad, 2H), 7.98 (d, 1H), 7.79 (dd, 1H), 7.75 (d, 1H), 7.32 (d, 2H), 7.12 (d, 2H), 6.97 (d, 1H), 6.90 (d, 2H), 6.88 (d, 2H), 4.93 (s, 2H), 4.59 (sep, 1H), 4.02 (m, 1H), 3.97 (t, 2H), 3.43 (s, 2H), 2.72-2.63 (3H), 2.11 (m, 1H), 1.97 (pseudo-quint, 2H), 1.83-1.74 (3H), 1.59 (m, 1H), 1.46 (m, 1H), 1.25 (d, 6H).

MS (ESI): m/z=690 (M+H⁺).

### Example 7

### Ethyl 4-(3-{4-[(4-isopropoxybenzyl)oxy]phenyl}propoxy)-3-(2-{[3-(methoxycarbonyl)-cyclohexyl]amino}-2-oxoethyl)benzoate

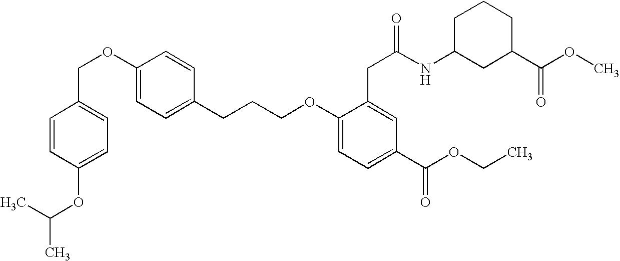

200 mg (0.39 mmol) of the compound from Example VI are introduced into 20 ml of dichloromethane, and 84 mg (0.43 mmol) of the compound from Example XXX are added. Then, in the stated sequence, 50 mg (0.39 mmol) of 1-hydroxy-1H-benzotriazole hydrate, 151 mg (0.79 mmol) of N′-(3-dimethylaminopropyl)-N-ethylcarbodiimide hydrochloride and 0.11 ml (0.79 mmol) of triethylamine are added. The reaction mixture is stirred at room temperature overnight. The mixture is evaporated to dryness, taken up in 3 ml of dimethyl sulfoxide and filtered. Subsequent purification by preparative RP-HPLC affords 175 mg of product.

HPLC (Method 1): Rₜ: 5.74 min.

¹H-NMR (300 MHz, DMSO-d₆, d/ppm): 7.88 (d, 1H), 7.81 (dd, 1H), 7.79 (d, 1H), 7.32 (d, 2H), 7.11 (d, 2H), 6.99 (d, 1H), 6.90 (d, 2H), 6.88 (d, 2H), 4.93 (s, 2H), 4.59 (sep, 1H), 4.28 (quart, 2H), 3.98 (t, 2H), 3.89 (m, 1H), 3.56 (s, 3H), 3.50 (s, 2H), 2.77-2.67 (3H), 1.97 (pseudo-quint, 2H), 1.79-1.38 (8H), 1.30 (t, 3H), 1.26 (d, 6H).

MS (ESI): m/z=646 (M+H⁺).

### Example 8

### 3-{2-[(3-Carboxycyclohexyl)amino]-2-oxoethyl}-4-(3-{4-8(4-isopropoxybenzyl)oxy]phenyl}-propoxy)benzoic acid

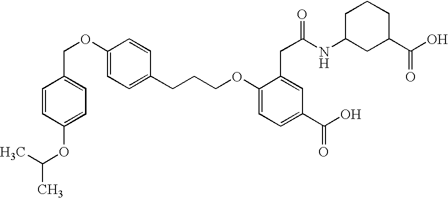

145 mg (0.22 mmol) of the compound from Example 7 are dissolved in 2 ml of tetrahydrofuran and 2 ml of methanol. The mixture is then mixed with 2 ml of 2 M sodium hydroxide solution and stirred at 60° C. for 1 hour. The reaction mixture is then freed of volatiles in a rotary evaporator, and 2.1 ml of 2 M hydrochloric acid are added to the aqueous residue. The product precipitates. It is filtered off with suction and washed with water. Drying under high vacuum results in 126 mg of product.

HPLC (Method 2): Rₜ: 4.95 min.

¹H-NMR (300 MHz, DMSO-d₆, d/ppm): 12.28 (s broad, 2H), 7.83 (d, 1H), 7.79 (dd, 1H), 7.77 (d, 1H), 7.32 (d, 2H), 7.12 (d, 2H) 6.97 (d, 1H), 6.90 (d, 2H), 6.88 (d, 2H), 4.93 (s, 2H), 4.59 (sep, 1H), 3.97 (t, 2H), 3.90 (m, 1H), 4.99 (s, 2H), 2.70 (t, 2H), 2.62 (m, 1H), 1.97 (pseudo-quint, 2H), 1.79-1.34 (8H), 1.24 (d, 6H).

MS (ESI): m/z=604 (M+H⁺).

### Example 9

### 3-{2-[2-(Carboxymethyl)pyrrolidin-1-yl]-2oxoethyl}-4-{3-[4-(4-phenoxybutoxy)phenyl]propoxy}-benzoic acid

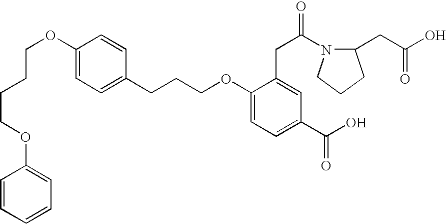

100 mg (0.20 mmol) of the compound from Example X are introduced into 4 ml of dichloromethane, and one drop of N,N-dimethylformamide is added. The solution is then cooled to 0° C., and 0.03 ml (0.30 mmol) of oxalyl chloride is added dropwise. The mixture is stirred at 0° C. for 15 minutes and then at room temperature for 1 hour. The solvent is stripped off in a rotary evaporator, and the residue is taken up in 4 ml of dichloromethane. 46.5 mg (0.30 mmol) of ethyl pyrrolidin-2-ylethanoate (Fukawa et al., Chem. Lett. 1982, 231-232) are added and stirred at room temperature for 1 hour. The generation of amide remains incomplete, however. The solvent is stripped off and taken up in methanol/tetrahydrofuran/water 2:2:1. Addition of 200 mg (3.56 mmol) of potassium hydroxide is followed by stirring at an oil bath temperature of 60° C. for 1 hour. The mixture is diluted with 20 ml of 0.5 M hydrochloric acid and extracted twice with ethyl acetate. After phase separation, the organic phase is dried, the solvent is stripped off, and the residue is purified by preparative RP-HPLC. Since the product is still impure it is subsequently purified on a normal phase by flash chromatography (mobile phase: dichloromethane/isopropanol 10:1→dichloromethane/methanol 5:1). 42 mg of the desired product are obtained.

LC-MS (Method 5): Rₜ: 2.56 min; MS (ESI+): m/z=590 (M+H⁺).

### Example 10

### Ethyl 3-{2-[(3-methoxy-3-oxopropyl)amino]-2-oxoethyl}-4-{3-[4-(4-phenoxybutoxy)phenyl]-propoxy}benzoate

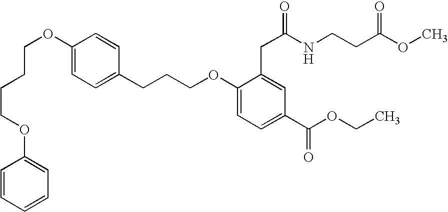

150 mg (0.29 mmol) of the compound from Example X are introduced into 3 ml of dichloromethane, 43.9 mg (0.31 mmol) of beta-alanine methyl ester hydrochloride are added and, at −20° C., 0.08 ml (0.57 mmol) of triethylamine are added dropwise. After the addition is complete, the reaction mixture is slowly brought to room temperature and stirred at this temperature for 2 hours. The mixture is mixed with 0.01 N hydrochloric acid and extracted with ethyl acetate. The organic phase is dried and concentrated. The residue is purified by preparative RP-HPLC. 40 mg of product are obtained.

¹H-NMR (200 MHz, DMSO-d₆, d/ppm): 8.04 (t, 1H), 7.74-7.89 (2H), 7.21-7.34 (2H), 7.06-7.18 (2H), 6.78-7.04 (6H), 4.27 (quart, 2H), 3.91-4.07 (6H), 3.56 (s, 3H), 3.47 (s, 2), 3.21-3.32 (2H), partly covered by water signal), 2.68 (t, 2H), 2.41-2.53 (2H, partly covered by DMSO signal), 1.96 (t, 2H), 1.79-1.90 (4H), 1.30 (t, 3H).

### Example 11

### 3-{2-[(2-Carboxyethyl)amino]-2-oxoethyl}-4-{3-[4-(4-phenoxybutoxy)phenyl]propoxy}benzoic acid

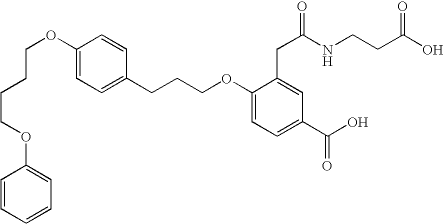

40 mg (0.07 mmol) of the compound from Example 10 are taken up in a 2:2:1 methanol/tetrahydrofuran/water mixture, mixed with 100 mg (1.78 mmol) of potassium hydroxide and stirred at room temperature overnight. The mixture is mixed with 20 ml of 0.5 M hydrochloric acid and extracted twice with ethyl acetate. After phase separation, the organic phase is dried and concentrated. 30 mg of product are obtained.

LC-MS (Method 7): Rₜ: 2.37 min; MS (ESI+): m/z=550 (M+H⁺).

¹H-NMR (200 MHz, DMSO-d₆, d/ppm): 12.40 (s, broad, 2H), 8.02 (t, 1H), 7.71-7.85 (2H), 7.21-7.34 (2H), 7.05-7.18 (2H), 6.78-7.04 (6H), 3.91-4.07 (6H), 3.46 (s, 2H), 3.24 (t, 2H), 2.68 (t, 2H), 2.38 (t, 2H), 1.96 (t, 2H), 1.79-1.90 (4H).

### Example 12

### Ethyl 3-{2-[(4-Ethoxy-4-oxobutyl)amino]-2-oxoethyl}-4-{3-[4-(4-phenoxybutoxy)phenyl]-propoxy}benzoate

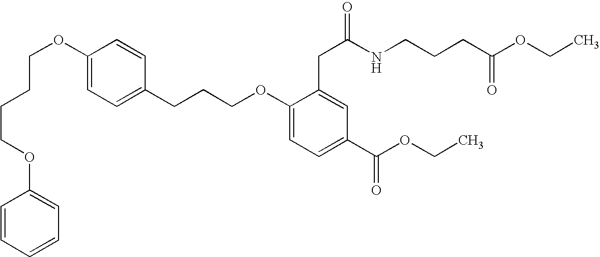

40 mg (0.08 mmol) of the compound from Example X, 13.2 mg (0.08 mmol) of ethyl 4-aminobutyrate hydrochloride, 60 mg (0.16 mmol) of O-(7-azabenzotriazol-1-yl)-N,N,N′N′-tetramethyluronium hexafluorophosphate and 0.04 ml (0.24 mmol) of N,N-diisopropylethylamine are dissolved in 2 ml of N,N-dimethylformamide and stirred at room temperature overnight. The mixture is then mixed with 1N hydrochloric acid, extracted with ethyl acetate, dried and concentrated in a rotary evaporator. 15 mg of product are obtained.

LC-MS (Method 8): Rₜ: 3.17 min; MS (ESI+): m/z=620 (M+H⁺).

¹H-NMR (200 MHz, DMSO-d₆, d/ppm): 7.97 (t, 1H), 7.71-7.87 (2H), 7.21-7.34 (2H), 7.06-7.16 (2H), 6.79-7.05 (6H), 4.27 (quart, 2H), 3.91-4.09 (8H), 3.48 (s, 2H), 3.00-3.14 (2H), 2.68 (t, 2H), 2.28 (t, 2H), 1.96 (t, 2H), 1.79-1.90 (4H), 1.65 (quint, 2H), 1.30 (t, 3H), 1.14 (t, 3H).

### Example 13

### 3-{2-[(3-Carboxypropyl)amino]-2-oxoethyl}-4-{3-[4-(4-phenoxybutoxy)phenyl]propoxy}benzoic acid

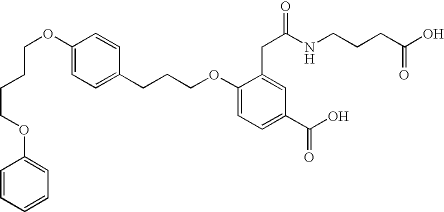

15 mg (0.02 mmol) of the compound from Example 12 are introduced into a 2:2:1 tetrahydrofuran/methanol/water mixture, mixed with 60 mg (1.07 mmol) of potassium hydroxide and stirred at room temperature for 4 hours. The mixture is acidified with 0.01 N hydrochloric acid and extracted with ethyl acetate. After phase separation, the organic phase is dried and concentrated. 18 mg of product are obtained.

LC-MS (Method 7): Rₜ: 2.38 min; MS (ESI+): m/z=564 (M+H⁺).

¹H-NMR (300 MHz, DMSO-d₆, d/ppm): 11.9-12.6 (s, broad, 2H), 7.91 (t, 1H), 7.74-7.83 (2H), 7.22-7.33 (2H), 7.07-7.16 (2H), 6.79-7.01 (6H), 3.94-4.06 (6H), 3.46 (s, 2H), 3.02-3.12 (2H), 2.68 (t, 2H), 2.21 (t, 2H), 1.97 (t, 2H), 1.81-1.89 (4H), 1.63 (quint, 2H).

### Example 14

### Ethyl 4-(3-{4-[4-(cyclohexyloxy)butoxy]phenyl}propoxy)-3-{2-[(4-methoxy-4-oxobutyl)amino]-2-oxoethyl}benzoate

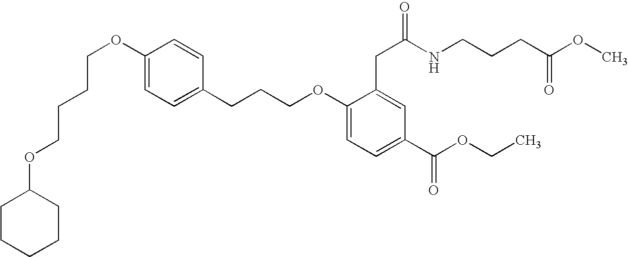

Under an argon atmosphere, 200 mg (0.39 mmol) of the compound from Example XIV are introduced into 20 ml of dichloromethane, and 50 mg (0.43 mmol) of methyl 4-aminobutyrate hydrochloride are added. Then, in the stated sequence, 50 mg (0.39 mmol) of 1-hydroxy-1H-benzotriazole hydrate, 149 mg (0.78 mmol) of N′-(3-dimethylaminopropyl)-N-ethylcarbodiimide hydrochloride and 0.11 ml (0.78 mmol) of triethylamine are added. The reaction mixture is stirred at room temperature overnight. The mixture is then evaporated to dryness, taken up in 3 ml of dimethyl sulfoxide and filtered, and the filtrate is purified by preparative RP-HPLC. The collected fractions afford 148 mg of product.

TLC: R_{f}: 0.28 (cyclohexane/ethyl acetate 1:1).

HPLC (Method 2): Rₜ: 5.80 min.

¹H-NMR (200 MHz, DMSO-d₆, d/ppm): 7.96 (t, 1H), 7.82 (dd, 1H), 7.79 (d, 1H), 7.11 (d, 2H), 6.99 (d, 1H), 6.81 (d, 2H), 4.28 (quart, 2H), 4.00-3.90 (4H), 3.55 (s, 3H), 3.47 (s, 2H), 3.41 (t, 2H), 3.21 (m, 2H), 3.07 (quart, 2H), 2.68 (t, 2H), 2.29 (t, 2H), 1.96 (pseudo-quint, 2H), 1.82-1.55 (9H), 1.48 (m, 1H), 1.29 (t, 3H), 1.25-1.13 (8H).

MS (ESI): m/z=612 (M+H⁺).

### Example 15

### 3-{2-[(3-Carboxypropyl)amino]-2-oxoethyl}-4-(3-{4-[4-(cyclohexyloxy)butoxy]phenyl}propoxy)-benzoic acid

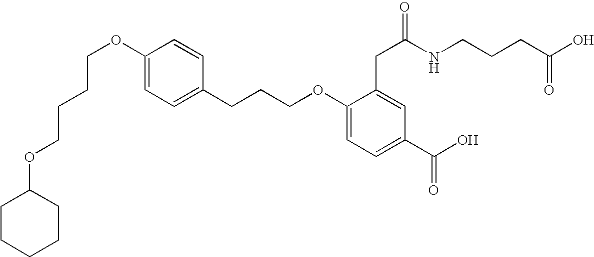

128 mg (0.21 mmol) of the compound from Example 14 are dissolved in 2 ml of tetrahydrofuran and 2 ml of methanol. The mixture is then mixed with 2 ml of 2 M sodium hydroxide solution and stirred at 60° C. for 1 hour. The reaction mixture is then freed of volatiles in a rotary evaporator, and 2.1 ml of 2 M hydrochloric acid are added to the aqueous residue. The product precipitates. It is filtered off with suction and washed with water. Drying under high vacuum results in 120 mg of product.

HPLC (Method 2): Rₜ: 4.97 min.

¹H-NMR (300 MHz, DMSO-d₆, d/ppm): 12.24 (s broad 2H), 7.91 (t, 1H), 7.79 (dd, 1H), 7.77 (d, 1H), 7.11 (d, 2H), 6.97 (d, 1H), 6.81 (d, 2H), 3.99-3.91 (4H), 3.46 (s, 2H), 3.41 (t, 2H), 3.22 (m, 2H), 3.07 (quart, 2H), 2.67 (t, 2H), 2.20 (t, 2H), 1.96 (pseudo-quint, 2H), 1.82-1.57 (9H), 1.43 (m, 1H), 1.25-1.13 (8H).

MS (ESI): m/z=570 (M+H⁺).

### Example 16

### Methyl 1-{[2-(3-{4-[4-(cyclohexyloxy)butoxy]phenyl}propoxy)-5-(ethoxycarbonyl)phenyl]-acetyl}piperidine-4-carboxylate

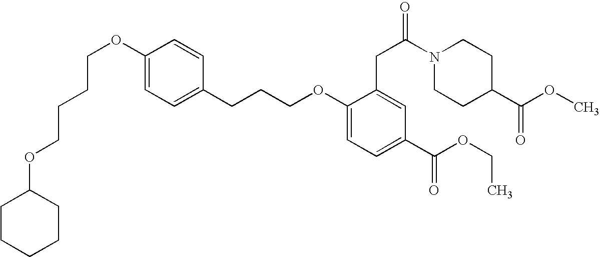

Under an argon atmosphere, 200 mg (0.39 mmol) of the compound from Example XIV are introduced into 20 ml of dichloromethane, and 61.5 mg (0.43 mmol) of methyl isonipectotate are added. Then, in the stated sequence, 50 mg (0.39 mmol) of 1-hydroxy-1H-benzotriazole hydrate, 150 mg (0.78 mmol) of N′-(3-dimethylaminopropyl)-N-ethylcarbodiimide hydrochloride and 0.11 ml (0.78 mmol) of triethylamine are added. The reaction mixture is stirred at room temperature overnight. The mixture is then evaporated to dryness, taken up in 3 ml of dimethyl sulfoxide and filtered, and the filtrate is purified by preparative RP-HPLC. The collected fractions afford 162 mg of product.

TLC: R_{f}: 0.35 (cyclohexane/ethyl acetate 1:1).

HPLC (Method 2): Rₜ: 6.08 min.

¹H-NMR (300 MHz, DMSO-₆, d/ppm): 7.82 (dd, 1H), 7.73 (d, 1H), 7.09 (d, 2H), 7.01 (d, 1H), 6.81 (d, 2H), 4.27 (quart, 2H), 4.24 (m, 1H), 4.01-3.88 (5H), 3.69 (m, 2H), 3.59 (s, 3H), 3.42 (t, 2H), 3.19 (m, 2H), 2.77-2.57 (4H), 1.95 (pseudo-quint, 2H), 1.83-1.70 (6H), 1.68-1.53 (4H), 1.52-1.38 (3H), 1.29 (t, 3H), 1.20 (5H).

MS (ESI): m/z=638 (M+N⁺).

### Example 17

### 1-{[5-Carboxy-2-(3-{4-[4-cyclohexyloxy)butoxy]phenyl}propoxy)phenyl]acetyl}piperidine-4-carboxylic acid

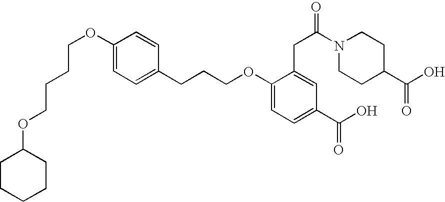

140 mg (0.22 mmol) of the compound from Example 16 are dissolved in 2 ml of tetrahydrofuran and 2 ml of methanol. The mixture is then mixed with 2 ml of 2 M sodium hydroxide solution and stirred at 60° C. for 1 hour. The reaction mixture is then freed of volatiles in a rotary evaporator, and 2.1 ml of 2 M hydrochloric acid are added to the aqueous residue. The solution is filtered with suction and the product washed with a little water. Drying under high vacuum results in 70 mg of product.

HPLC (Method 2): Rₜ: 5.13 min.

¹H-NMR (400 MHz, DMSO-d₆, d/ppm): 7.79 (dd, 1H), 7.71 (d, 1H), 7.10 (d, 2H), 6.97 (d, 1H), 6.82 (d, 2H), 4.21 (m, 1H), 4.97-3.89 (5H), 3.67 (m, 2H), 3.20 (m, 2H), 3.10 (m, 2H), 2.72 (t, 1H), 2.63 (t, 2H), 2.48 (m, 1H, partly concealed by DMSO signal), 1.94 (pseudo-quint, 2H), 1.83-1.69 (6H), 1.64-1.56 (4H), 1.49-1.31 (3H), 1.19 (5H).

MS (ESI): m/z=596 (M+H⁺).

### Example 18

### Ethyl 4-(3-{4-[4-(cyclohexyloxy)butoxy]phenyl}propoxy)-3-{2-[(3-ethoxy-3-oxopropyl)amino]-2-oxoethyl}benzoate

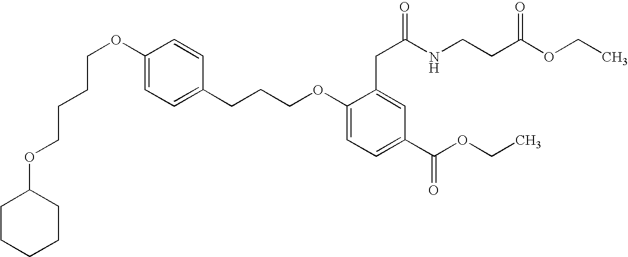

150 mg (0.29 mmol) of the compound from Example XIV are introduced into 2 ml of dichloromethane. In the stated sequence, firstly 84 mg (0.44 mmol) of N′-(3-dimethylaminopropyl)-N-ethylcarbodiimide hydrochloride, 59 mg (0.44 mmol) of 1-hydroxy-1H-benzotriazole hydrate and then 0.15 ml (0.88 mmol) of N,N-diisopropylethylamine are added. Then 49 mg (0.32 mmol) of beta-alanine ethyl ester hydrochloride are dissolved in 2 ml of N,N-dimethylformamide and added dropwise. The reaction mixture is stirred at room temperature overnight. For working up, the mixture is diluted with dichloromethane and washed with water. The organic phase is washed once each with saturated sodium bicarbonate solution and saturated sodium chloride solution, dried over magnesium sulfate, filtered and concentrated. The residue is purified on a silica gel column (mobile phase: cyclohexane/ethyl acetate 4:1→3:1). 90 mg of product are isolated.

TLC: R_{f}: 0.46 (cyclohexane/ethyl acetate 1:1).

HPLC (Method 9): Rₜ: 5.61 min.

¹H-NMR (300 MHz, DMSO-d₆, d/ppm): 7.96 (t, 1H), 7.82 (dd, 1H), 7.77 (d, 1H), 7.11 (d, 2H), 7.00 (d, 1H), 6.83 (d, 2H), 4.27 (q, 2H), 4.07-3.89 (m, 6H), 3.49-3.38 (m, 4H), 3.25-3.15 (m, 2H), 2.67 (t, 2H), 2.44 (t, 2H), 2.11-1.90 (m, 2H), 1.84-1.55 (m, 8H), 1.50-1.40 (m, 1H), 1.30 (t, 3H), 1.26-1.11 (m, 9H).

LC-MS (Method 7): Rₜ: 3.17 min, m/z=611 (M+H⁺).

### Example 19

### 3-{2-[(2-Carboxyethyl)amino]-2-oxoethyl}-4-(3-{4-[4-(cyclohexyloxy)butoxy]phenyl}propoxy)-benzoic acid

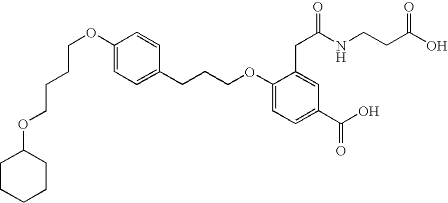

63 mg (0.10 mmol) of the compound from Example 18 and 80 mg (2.00 mmol) of 2 M sodium hydroxide solution are taken up in 1 ml of methanol and 1 ml of tetrahydrofuran and heated at 60° C. for 1 hour. The methanol and the tetrahydrofuran are removed in a rotary evaporator, and the residue is mixed with a little water. While cooling in ice, it is then acidified with 1 M hydrochloric acid solution. The product precipitates. It is filtered off with suction, washed with water and dried under high vacuum at 40° C. overnight. 48 mg of product are obtained.

HPLC (Method 9): Rₜ: 4.74 min

¹H-NMR (200 MHz, DMSO-d₆, d/ppm): 12.5 (s, broad, 2H), 7.98 (t, 1H), 7.79 (dd, 2H), 7.11 (d, 2H), 6.96 (d, 1H), 6.81 (d, 2H), 3.95 (q, 4H), 3.48-3.17 (m, 7H), 2.68 (t, 2H), 2.36 (t, 2H), 2.02-1.90 (m, 2H), 1.83-1.52 (m, 9H), 1.30-1.11 (m, 5H).

MS (ESI): m/z=556 (M+H⁺), 578 (M+Na⁺).

### Example 20

### Methyl 1-{[2-(3-{4-[4-(cyclohexyloxy)butoxy]phenyl}propoxy)-5-(ethoxycarbonyl)phenyl]-acetyl}azetidine-3-carboxylate

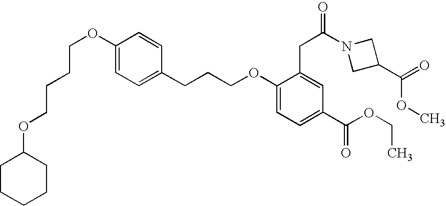

150 mg (0.29 mmol) of the compound from Example XIV are introduced into 2 ml of dichloromethane. In the stated sequence, firstly 84 mg (0.44 mmol) of N′-(3-dimethylamino-propyl)-N-ethylcarbodiimide hydrochloride, 59 mg (0.44 mmol) of 1-hydroxy-1H-benzotriazole hydrate and then 0.15 ml (0.88 mmol) of N,N-diisopropylethylamine are added. Then 53 mg (0.35 mmol) of the compound from Example XV are dissolved in 2 ml of N,N-dimethylformamide and added dropwise. The reaction mixture is stirred at room temperature overnight. For working up, the mixture is diluted with dichloromethane and washed with water. The organic phase is washed once each with saturated sodium bicarbonate solution and saturated sodium chloride solution, dried over magnesium sulfate, filtered and concentrated. The residue is purified on a silica gel column (mobile phase: cyclohexane/ethyl acetate 3:1). 108 mg of product are isolated.

TLC: R_{f}: 0.31 (cyclohexane/ethyl acetate 1:1).

HPLC (Method 9): Rₜ: 5.55 min.

¹H-NMR (300 MHz, DMSO-d₆, d/ppm): 7.83 (dd, 1H), 7.79 (d, 1H), 7.11 (d, 2H), 7.01 (d, 1H), 6.82 (d, 2H), 4.38 (t, 1H), 4.27 (q, 3H), 4.06-3.97 (m, 3H), 3.96-3.86 (m, 3H), 3.64 (s, 3H), 3.54-3.39 (m, 4H), 3.25-3.15 (m, 1H), 2.67 (t, 2H), 2.04-1.05 (m, 2H),1.83-1.55 (m, 8H), 1.50-1.40 (m, 1H), 1.30 (t, 3H), 1.27-1.14 (m, 6H).

LC-MS (Method 7): Rₜ: 3.13 min, m/z=609 (M+H⁺).

### Example 21

### 1-{[5-Carboxy-2-(3-{4-[4-(cyclohexyloxy)butoxy]phenyl}propoxy)phenyl]acetyl}azetidine-3-carboxylic acid

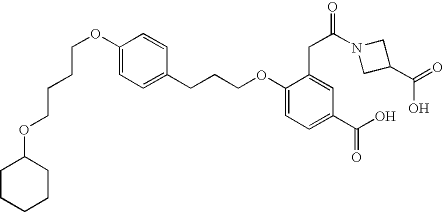

78 mg (0.13 mmol) of the compound from Example 20 and 1 ml (2.00 mmol) of 2 M sodium hydroxide solution are taken up in 1 ml of methanol and 1 ml of tetrahydrofuran and heated at 60° C. for 1 hour. After cooling to room temperature, the methanol and the tetrahydrofuran are removed in a rotary evaporator, and the residue is mixed with a little water. While cooling in ice, it is then acidified with 1 M hydrochloric acid. The crystals which separate out are filtered off with suction, washed with water and dried under high vacuum at 40° C. overnight. 51.6 mg of product are obtained.

HPLC (Method 9): Rₜ: 4.74 min.

¹H-NMR (200 MHz, DMSO-d₆, d/ppm): 12.65 (s, broad, 2H), 7.84-7.76 (m, 2H), 7.11 (d, 2H), 6.88 (d, 1H), 6.82 (d, 2H), 4.41-4.20 (m, 2H), 4.08-3.82 (m, 6H), 3.49-3.13 (m, 6H), 2.67 (t, 2H), 2.07-1.90 (m, 2H), 1.88-1.51 (m, 9H), 1.30-1.11 (m, 5H).

MS (ESI): m/z=568 (M+H⁺), 590 (M+Na⁺).

### Example 22

### Ethyl 4-(3-{4-[4-(cyclohexyloxy)butoxy]phenyl}propoxy)-3-{2-[(2-methoxy-2-oxoethyl)amino]-2-oxoethyl}benzoate

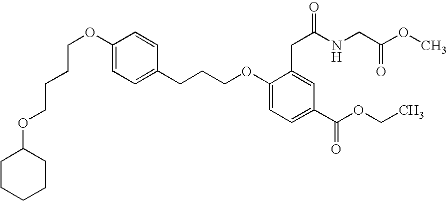

Under an argon atmosphere, 150 mg (0.29 mmol) of the compound from Example XIV are introduced into 15 ml of dichloromethane, and 40.4 mg (0.32 mmol) of glycine methyl ester hydrochloride are added. Then, in the stated sequence, 39.5 mg (0.29 mmol) of 1-hydroxy-1H-benzotriazole hydrate, 112.2 mg (0.59 mmol) of N′-(3-dimethylaminopropyl)-N-ethylcarbodiimide hydrochloride and 0.12 ml (0.88 mmol) of triethylamine are added. The reaction mixture is stirred at room temperature overnight. The mixture is mixed with 200 ml of water and extracted three times with ethyl acetate, and the combined organic phases are washed with saturated sodium chloride solution, dried and concentrated in a rotary evaporator. The residue is separated by preparative RP-HPLC. 116 mg of product are obtained.

TLC: R_{f}: 0.83 (ethyl acetate/methanol 1:1).

HPLC (Method 2): Rₜ: 5.76 min.

¹H-NMR (400 MHz, DMSO-d₆, d/ppm): 8.37 (t, 1H), 7.83-7.81 (2H), 7.11 (d, 2H), 7.01 (d, 1H), 6.82 (d, 2H), 4.27 (q, 2H), 3.98 (t, 2H), 3.92 (t, 2H), 3.83 (d, 2H), 3.60 (s, 3H), 3.55 (s, 2H), 3.42 (t, 2H), 3.20 (m, 1H), 2.67 (t, 2H), 1.97 (pseudo-quint., 2H), 1.79 (m, 2H), 1.71 (m, 2H), 1.67-1.56 (4H), 1.96 (m, 1H), 1.30 (t, 3H), 1.23-1.13 (5H).

MS (ESI): m/z=584 (M+H⁺).

### Example 23

### 3-{2-[(Carboxymethyl)amino]-2-oxoethyl}-4-(3-{4-[4-(cyclohexyloxy)butoxy]phenyl}propoxy)-benzoic acid

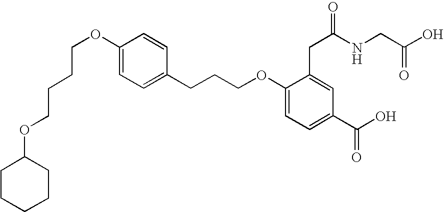

91 mg (0.16 mmol) of the compound from Example 22 are dissolved in 4 ml of ethanol, mixed with 1.25 ml of 1M sodium hydroxide solution and stirred at room temperature for 1 hour and 15 minutes. Then, at room temperature, 0.107 ml (1.87 mmol) of glacial acetic acid is added until the solution has a pH of 10. Subsequently, a total of 0.244 ml of 32% strength hydrochloric acid solution is gradually added until a pH of 1-2 is reached. The product precipitates. Some of the solvent is stripped off in a rotary evaporator, and the residue is filtered off with suction and washed twice with water. The product obtained in this way is dried in vacuo at 40° C. for 5 hours. 34 mg of product are obtained.

HPLC (Method 2): Rₜ: 5.04 min.

¹H-NMR (300 MHz, DMSO-d₆, d/ppm): 12.48 (s broad, 2H), 8.16 (t, 1H), 7.82-7.79 (2H), 7.11 (d, 2H), 6.98 (d, 1H), 6.82 (d, 2H), 3.98 (t, 2H), 3.92 (t, 2H), 3.76 (d, 2H), 3.53 (s, 2H), 3.42 (t, 2H), 3.20 (m, 1H), 2.67 (t, 2H), 1.97 (pseudo-quint., 2H), 1.82-1.54 (8H), 1.47 (m, 1H), 1.25-1.16 (5H).

MS (ESI): m/z=542 (M+H⁺).

### Example 24

### Ethyl 1-{[2-(3-{4-[4-(cyclohexyloxy)butoxy]phenyl}propoxy)-5-(ethoxycarbonyl)phenyl]acetyl}-piperidine-3-carboxylate

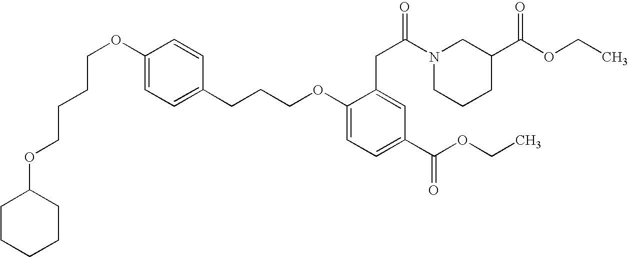

Under an argon atmosphere, 150 mg (0.29 mmol) of the compound from Example XIV are introduced into 15 ml of dichloromethane, and 50.6 mg (0.32 mmol) of ethyl 3-piperidine-carboxylate are added. Then, in the stated sequence, 39.5 mg (0.29 mmol) of 1-hydroxy-1H-benzotriazole hydrate, 112.2 mg (0.59 mmol) of N′-(3-dimethylaminopropyl)-N-ethylcarbodiimide hydrochloride and 0.08 ml (0.59 mmol) of triethylamine are added. The reaction mixture is stirred at room temperature overnight. The mixture is mixed with 200 ml of water and extracted three times with ethyl acetate, and the combined organic phases are washed with saturated sodium chloride solution, dried and concentrated in a rotary evaporator. The residue is separated by preparative RP-HPLC. 164 mg of product are obtained.

LC/MS (Method 7): Rₜ: 3.32 min; m/z=652 (M+H⁺).

### Example 25

### 1-{[5-Carboxy-2-(3-{4-[4-(cyclohexyloxy)butoxy]phenyl}propoxy)phenyl]acetyl}piperidine-3-carboxylic acid

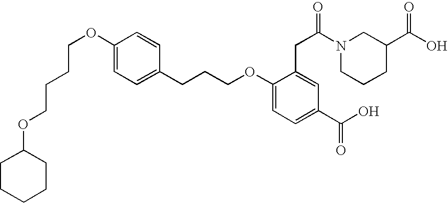

140 mg (0.21 mmol) of the compound from Example 24 are dissolved in 4 ml of ethanol, mixed with 1.7 ml of 1 M sodium hydroxide solution and stirred at room temperature for 1 hour. Then, at room temperature, 0.15 ml (2.58 mmol) of glacial acetic acid is added until the solution has a pH of 10. Subsequently, a total of 0.33 ml of 32% strength hydrochloric acid is added until a pH of 1-2 is reached. The mixture is extracted three times with ethyl acetate. The combined organic phase is washed once with saturated sodium chloride solution, dried and concentrated in a rotary evaporator. The residue is dried under high vacuum and subsequently separated by preparative RP-HPLC. 48 mg of product are obtained.

LC/MS (Method 4): Rₜ: 2.80 min; m/z=596 (M+H⁺).

### Example 26

### Ethyl 4-(3-{4-[4-(cyclohexyloxy)butoxy]phenyl}propoxy)-3-(2-{[(1S,3R)-3-(methoxycarbonyl)-cyclopentyl]amino}-2-oxoethyl)benzoate

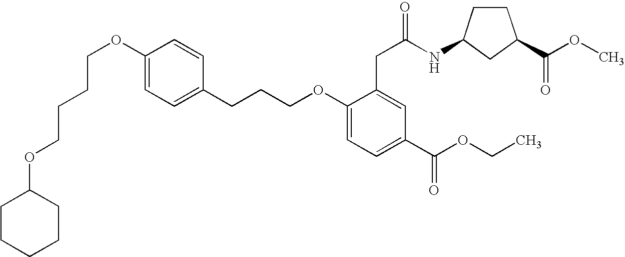

Under an argon atmosphere, 200 mg (0.39 mmol) of the compound from Example XIV are introduced into 20 ml of dichloromethane, and 77.1 mg (0.43 mmol) of the compound from Example VII are added. Then, in the stated sequence, 50 mg (0.39 mmol) of 1-hydroxy-1H-benzotriazole hydrate, 149.6 mg (0.78 mmol) of N′-(3-dimethylaminopropyl)-N-ethylcarbodiimide hydrochloride and 0.11 ml (0.78 mmol) of triethylamine are added. The reaction mixture is stirred at room temperature overnight. The mixture is then evaporated to dryness, taken up in 3 ml of dimethyl sulfoxide and filtered, and the filtrate is purified by preparative HPLC. The collected fractions afford 187 mg of product.

TLC: R_{f}: 0.29 (cyclohexane/ethyl acetate 1:1).

HPLC (Method 2): Rₜ: 5.89 min.

¹H-NMR (200 MHz, DMSO-d₆, d/ppm): 8.04 (d, 1H), 7.82 (dd, 1H), 7.77 (d, 1H), 7.11 (d, 2H), 6.99 (d, 1H), 6.81 (d, 2H), 4.28 (quart, 2H), 4.09-3.89 (4H), 3.58 (s, 3H), 3.45 (s, 2H), 3.41 (t, 2H), 3.20 (m, 1H), 2.86-2.63 (3H), 2.12 (m, 1H), 1.96 (m, 2H), 1.85-1.41 (15H), 1.30 (t, 3H), 1.19 (5H).

MS (ESI): m/z=638 (M+H⁺).

### Example 27

### 3-(2-{[(1S,3R)-3-Carboxycyclopentyl]amino}-2-oxoethyl)-4-(3-{4-[4-(cyclohexyloxy)butoxy]-phenyl}propoxy)benzoic acid

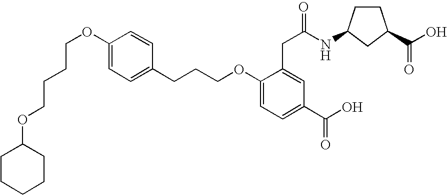

170 mg (0.27 mmol) of the compound from Example 26 are dissolved in 2 ml of tetrahydrofuran and 2 ml of methanol. The mixture is then mixed with 2 ml of 2 M sodium hydroxide solution and stirred at 60° C. for 1 hour. The reaction mixture is then freed of volatiles in a rotary evaporator, and 2.1 ml of 2 M hydrochloric acid are added to the aqueous residue. The product precipitates. It is filtered off with suction and washed with water. Drying under high vacuum results in 94 mg of product.

HPLC (Method 3): Rₜ: 5.07 min.

¹H-NMR (300 MHz, DMSO-d₆, d/ppm): 7.97 (d, 1H), 7.78 (dd, 1H), 7.75 (d, 1H), 7.11 (d, 2H), 6.95 (d, 1H), 6.81 (d, 2H), 4.06-3.90 (5H), 2.69 (3H), 2.19 (m, 1H), 1.96 (pseudo-quint, 2H), 1.83-1.55 (14H), 1.45 (m, 2H), 1.19 (5H).

MS (ESI): m/z=596 (M+H⁺).

### Example 28

### Ethyl 4-(3-{4-[4-(cyclohexyloxy)butoxy]phenyl}propoxy)-3-(2-{[3-(methoxycarbonyl)-cyclohexyl]amino}-2-oxoethyl)benzoate

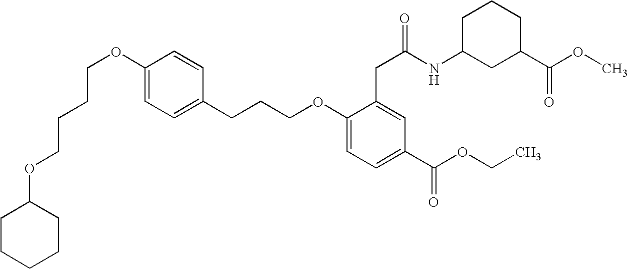

Under an argon atmosphere, 200 mg (0.39 mmol) of the compound from Example XIV are introduced into 20 ml of dichloromethane, and 83.1 mg (0.43 mmol) of the compound from Example XXX are added. Then, in the stated sequence, 50.0 mg (0.39 mmol) of 1-hydroxy-1H-benzotriazole hydrate, 149.6 mg (0.78 mmol) of N′-(3-dimethylaminopropyl)-N-ethylcarbodiimide hydrochloride and 0.11 ml (0.78 mmol) of triethylamine are added. The reaction mixture is stirred at room temperature overnight. The mixture is then evaporated to dryness, taken up in 3 ml of dimethyl sulfoxide and filtered. Subsequent purification by preparative RP-HPLC affords 170.9 mg of product.

HPLC (Method 2): Rₜ: 6.12 min.

¹H-NMR (300 MHz, DMSO-d₆, d/ppm): 7.88 (d, 1H), 7.81 (dd, 1H), 7.78 (d, 1H), 7.11 (d, 2H), 6.99 (d, 1H), 6.81 (d, 2H), 4.27 (quart, 2H), 4.00-3.85 (5H), 3.57 (s, 3H), 3.50 (s, 2H), 3.42 (t, 2), 3.21 (m, 1H), 2.76-2.67 (3H), 1.97 (pseudo-quint, 2H), 1.82-1.38 (17H), 1.29 (t, 3), 1.27-1.15 (5H).

MS (ESI): m/z=652 (M+H⁺).

### Example 29

### 3-{2-[(3-Carboxycyclohexyl)amino]-2-oxoethyl}-4-(3-{4-[4-(cyclohexyloxy)butoxy]phenyl}-propoxy)benzoic acid

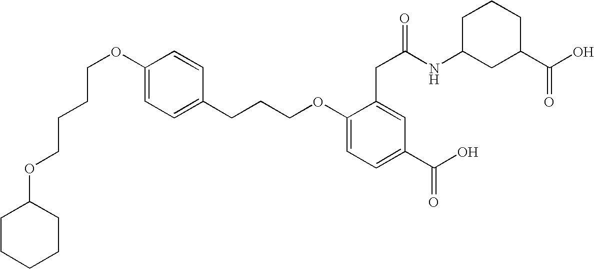

146 mg (0.22 mmol) of the compound from Example 28 are dissolved in 2 ml of tetrahydrofuran and 2 ml of methanol. The mixture is then mixed with 2 ml of 2 M sodium hydroxide solution and stirred at 60° C. for 1 hour. The reaction mixture is then freed of volatiles in a rotary evaporator, and 2.1 ml of 2 M hydrochloric acid are added to the aqueous residue. The product precipitates. It is filtered off with suction and washed with water. Drying under high vacuum results in 112 mg of product.

HPLC (Method 2): Rₜ: 5.19 min.

¹H-NMR (300 MHz, DMSO-d₆, d/ppm): 12.28 (s broad, 2H), 7.83 (d, 1H), 7.80 (dd, 1H), 7.77 (d, 1H), 7.11 (d, 2H), 6.96 (d, 1H), 6.81 (d, 2H), 3.99-3.87 (5H), 3.49 (s, 2H), 3.42 (t, 2H), 3.20 (m, 1H), 2.69 (t, 2H), 2.62 (m, 1H), 1.98 (pseudo-quint, 2H), 1.82-1.37 (17H), 1.28-1.13 (5H).

MS (ESI): m/z=610 (M+H⁺).

### Example 30

### Ethyl 4-{3-[4-(3-cyclohexylpropoxy)phenyl]propoxy}-3-{2-[(4-methoxy-4-oxobutyl)amino]-2-oxoethyl}benzoate

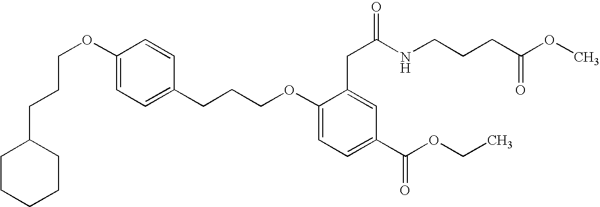

Under an argon atmosphere, 200 mg (0.41 mmol) of the compound from Example XVII are introduced into 20 ml of dichloromethane, and 53.4 mg (0.46 mmol) of methyl 4-aminobutyrate hydrochloride are added. Subsequently, in the stated sequence, 60 mg (0.41 mmol) of 1-hydroxy-1H-benzotriazole hydrate, 158.9 mg (0.83 mmol) of N′-(3-dimethylaminopropyl)-N-ethyl-carbodiimide hydrochloride and 0.12 ml (0.83 mmol) of triethylamine are added. The reaction mixture is stirred at room temperature overnight. The mixture is evaporated to dryness, taken up in 3 ml of dimethyl sulfoxide and filtered. Subsequent purification of the filtrate by preparative RP-HPLC affords 162.3 mg of product.

TLC: R_{f}: 0.29 (cyclohexane/ethyl acetate 1:1).

HPLC (Method 2): Rₜ: 6.25 min.

¹H-NMR (300 MHz, DMSO-d₆, d/ppm): 7.92 (t, 1H), 7.81 (dd, 1H), 7.78 (d, 1H), 7.10 (d, 2H), 6.99 (d, 1H), 6.81 (d, 2H), 4.28 (quart, 2H), 3.98 (t, 2H), 3.88 (t, 2H), 3.55 (s, 3H), 3.47 (s, 2H), 3.07 (t, 2H), 2.67 (t, 2H), 2.29 (t, 2H), 1.96 (pseudo-quint, 2H), 1.72-1.57 (9H), 1.30 (t, 3H), 1.31-1.09 (6H), 0.87 (m, 2H).

MS (ESI): m/z=582 (M+H⁺).

### Example 31

### 3-{2-[(3-Carboxypropyl)amino]-2-oxoethyl}-4-{3-[4-(3-cyclohexylpropoxy)phenyl]propoxy}-benzoic acid

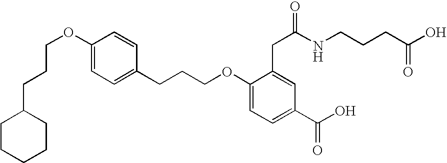

142 mg (0.24 mmol) of the compound from Example 30 are dissolved in 2 ml of tetrahydrofuran and 2 ml of methanol. The mixture is then mixed with 2 ml of 2 M sodium hydroxide solution and stirred at 60° C. for 1 hour. The reaction mixture is then freed of volatiles in a rotary evaporator, and 2.1 ml of 2 M hydrochloric acid are added to the aqueous residue. The product precipitates. It is filtered off with suction and washed with water. Drying under high vacuum results in 130 mg of product.

HPLC (Method 2): Rₜ: 5.35 min.

¹H-NMR (300 MHz, DMSO-d₆, d/ppm): 12.23 (s broad, 2H), 7.91 (t, 1H), 7.79 (dd, 1H), 7.76 (d, 1H), 7.10 (d, 2H), 6.96 (d, 1H), 6.81 (d, 2H), 3.97 (t, 2H), 3.88 (t, 2H), 3.45 (s, 2H), 3.07 (quart, 2H), 2.68 (t, 2H), 2.20 (t, 2H), 1.97 (pseudo-quint, 2H), 1.73-1.58 (9H), 1.31-1.04 (6H), 0.87 (m, 2H).

MS (ESI): m/z=540 (M+H⁺).

### Example 32

### Methyl 1-{[2-{3-[4-(3-cyclohexylpropoxy)phenyl]propoxy}-5-(ethoxycarbonyl)phenyl]acetyl}-piperidine-4-carboxylate

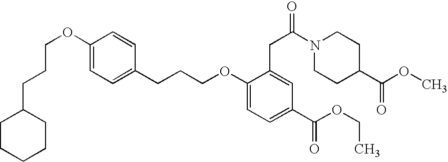

Under an argon atmosphere, 200 mg (0.41 mmol) of the compound from Example XVII are introduced into 20 ml of dichloromethane, and 65.3 mg (0.46 mmol) of methyl isonipectotate are added. Subsequently, in the stated sequence, 60 mg (0.41 mmol) of 1-hydroxy-1H-benzotriazole hydrate, 158.9 mg (0.83 mmol) of N′-(3-dimethylaminopropyl)-N-ethylcarbodiimide hydrochloride and 0.12 ml (0.83 mmol) of triethylamine are added. The reaction mixture is stirred at room temperature overnight. The mixture is evaporated to dryness, taken up in 3 ml of dimethyl sulfoxide and filtered. The filtrate is purified by preparative HPLC. 197.6 mg of product are obtained.

TLC: R_{f}: 0.37 (cyclohexane/ethyl acetate 1:1).

HPLC (Method 2): Rₜ: 6.68 min.

¹H-NMR (300 MHz, DMSO-d₆, d/ppm): 7.82 (dd, 1H), 7.73 (d, 1H), 7.08 (d, 2H), 7.01 (d, 1H), 6.80 (d, 2H), 4.27 (quart, 2H), 4.23 (m, 1H), 3.98 (t, 2H), 3.93 (m, 1H), 3.88 (t, 2H), 3.69 (m, 2H), 3.58 (s, 3H), 3.14 (m, 2H), 2.77-2.58 (4H), 1.94 (pseudo-quint, 2H), 1.82 (m, 2H), 1.82 (m, 2H), 1.71-1.58 (6H), 1.52-1.08 (11H), 0.87 (m, 2H).

MS (ESI): m/z=608 (M+H⁺).

### Example 33

### 1-[(5-Carboxy-2-{3-[4-(3-cyclohexylpropoxy)phenyl]propoxy}phenyl)acetyl]piperidine-4-carboxylic acid

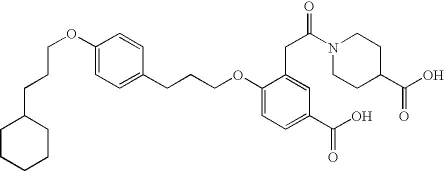

180 mg (0.30 mmol) of the compound from Example 32 are dissolved in 2 ml of tetrahydrofuran and 2 ml of methanol. The mixture is then mixed with 2 ml of 2 M sodium hydroxide solution and stirred at 60° C. for 1 hour. The reaction mixture is freed of volatiles in a rotary evaporator, and then 2.1 ml of 2 M hydrochloric acid are added. The product precipitates. It is filtered off with suction and washed with water. Drying under high vacuum results in 119.1 mg of product.

HPLC (Method 2): Rₜ: 5.48 min.

¹H-NMR (300 MHz, DMSO-d₆, d/ppm): 12.36 (s broad, 2H), 7.80 (dd, 1H), 7.72 (d, 1H), 7.08 (d, 2H), 6.98 (d, 1H), 6.81 (d, 2H), 4.22 (m, 1H), 3.97 (t, 2H), 3.92 (m, 1H), 3.88 (t, 2H), 3.67 (m, 2H), 3.12 (m, 2H), 2.77-2.61 (4H), 1.95 (pseudo-quint, 2H), 1.80 (m, 2H), 1.72-1.58 (6H), 1.51-1.09 (8H), 0.87 (m, 2H).

MS (ESI): m/z=566 (M+H⁺).

### Example 34

### Ethyl 4-{3-[4-(3-cyclohexylpropoxy)phenyl]propoxy}-3-(2-{[(1S,3R)-3-(methoxycarbonyl)-cyclopentyl]amino}-2-oxoethyl)benzoate

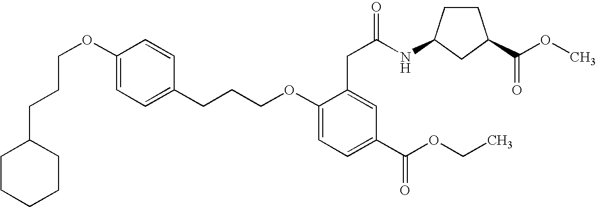

Under an argon atmosphere, 200 mg (0.41 mmol) of the compound from Example XVII are introduced into 20 ml of dichloromethane, and 81.9 mg (0.46 mmol) of the compound from Example VII are added. Subsequently, in the stated sequence, 56 mg (0.41 mmol) of 1-hydroxy-1H-benzotriazole hydrate 158.9 mg (0.83 mmol) of N′-(3-dimethylaminopropyl)-N-ethyl-carbodiimide-hydrochloride and 0.12 ml (0.83 mmol) of triethylamine are added. The reaction mixture is stirred at room temperature overnight. The mixture is then evaporated to dryness, taken up in 3 ml of dimethyl sulfoxide, filtered and purified by preparative RP-HPLC. The collected fractions afford 151.6 mg of product.

TLC: R_{f}: 0.33 (cyclohexane/ethyl acetate 1:1).

HPLC (Method 2): Rₜ: 6.40 min.

¹H-NMR (200 MHz, DMSO-d₆, d/ppm): 8.04 (d, 1H), 7.82 (dd, 1H), 7.78 (d, 1H), 7.11 (d, 2H), 6.98 (d, 1H), 6.81 (d, 2H), 4.27 (quart, 2H), 4.08-3.84 (5H), 3.58 (s, 3H), 3.44 (s, 2H), 2.82-2.63 (3H), 2.12 (m, 1H), 1.96 (pseudo-quint, 2H), 1.82-1.58 (12H), 1.32-1.11 (9H), 0.88 (m, 2H).

MS (ESI): m/z=608 (M+H⁺).

### Example 35

### 3-(2-{[(1S,3R)-3-Carboxycyclopentyl]amino}-2-oxoethyl)-4-{3-[4-(3-cyclohexylpropoxy)-phenyl]propoxy}benzoic acid

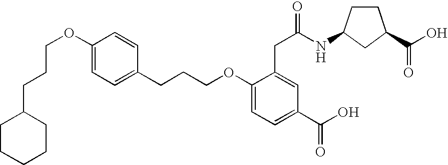

130 mg (0.21 mmol) of the compound from Example 34 are dissolved in 2 ml of tetrahydrofuran and 2 ml of methanol. The mixture is then mixed with 2 ml of 2 M sodium hydroxide solution and stirred at 60° C. for 1 hour. The reaction mixture is freed of volatiles in a rotary evaporator, and then 2.1 ml of 2 M hydrochloric acid are added. The product precipitates. It is filtered off with suction and washed with water. Drying under high vacuum results in 65.7 mg of product.

HPLC (Method 2): Rₜ: 5.39 min.

¹H-NMR (300 MHz, DMSO-d₆, d/ppm): 12.27 (s broad, 2H), 7.98 (d, 1H), 7.79 (dd, 1H), 7.74 (d, 1H), 7.11 (d, 2H), 6.96 (d, 1H), 6.81 (d, 2H), 4.02 (m, 1H), 3.96 (t, 2H), 3.88 (t, 2H), 3.43 (s, 2H), 2.72-2.62 (3H), 2.11 (m, 1H), 1.96 (pseudo-quint, 2H), 1.83-1.56 (12H), 1.31-1.11 (6), 0.87 (m, 2H).

MS (ESI): m/z=566 (M+H⁺).

### Example 36

### Ethyl 4-{3-[4-(3-cyclohexylpropoxy)phenyl]propoxy}-3-(2-{[3-(methoxycarbonyl)cyclohexyl]-amino}-2-oxoethyl)benzoate

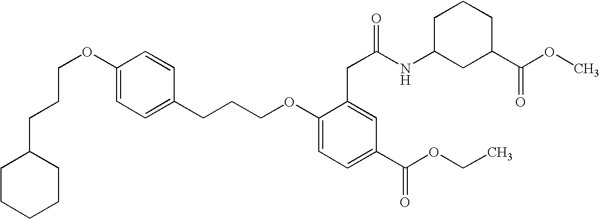

Under an argon atmosphere, 200 mg (0.41 mmol) of the compound from Example XVII are introduced into 20 ml of dichloromethane, and 88.3 mg (0.46 mmol) of the compound from Example XXX are added. Subsequently, in the stated sequence, 56 mg (0.41 mmol) of 1-hydroxy-1H-benzotriazole hydrate 158.9 mg (0.83 mmol) of N′-(3-dimethylaminopropyl)-N-ethylcarbodiimide-hydrochloride and 0.12 ml (0.83 mmol) of triethylamine are added. The reaction mixture is stirred at room temperature overnight. The mixture is then evaporated to dryness, taken up in 3 ml of dimethyl sulfoxide, filtered and purified by preparative RP-HPLC. The collected fractions afford 163.7 mg of product.

HPLC (Method 2): Rₜ: 6.70 min.

¹H-NMR (300 MHz, DMSO-d₆, d/ppm): 7.88 (d, 1H), 7.81 (dd, 1H), 7.78 (d, 1H), 7.10 (d, 2), 6.99 (d, 1H), 6.81 (d, 2H), 4.27 (quart, 2H), 3.97 (t, 2H), 3.91-3.86 (3H), 3.56 (s, 3H), 3.50 (s, 2H), 2.77-2.66 (3H), 1.97 (pseudo-quint, 2H), 1.73-1.38 (14H), 1.32-1.12 (10H), 0.87 (m, 2H).

MS (ESI): m/z=622 (M+H⁺).

### Example 37

### 3-{2-[(3-Carboxycyclohexyl)amino]-2-oxoethyl}-4-{3-[4-(3-cyclohexylpropoxy)phenyl]propoxy}-benzoic acid

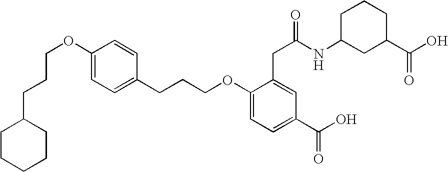

140 mg (0.23 mmol) of the compound from Example 36 are dissolved in 2 ml of tetrahydrofuran and 2 ml of methanol. The mixture is then mixed with 2 ml of 2 M sodium hydroxide solution and stirred at 60° C. for 1 hour. The reaction mixture is then freed of volatiles in a rotary evaporator and extracted with diethyl either. 2.1 ml of 2 M hydrochloric acid are added to the aqueous residue. The product precipitates. It is filtered off with suction and washed with water. Drying under high vacuum results in 125.3 mg of product.

HPLC (Method 2): Rₜ: 5.57 min.

¹H-NMR (300 MHz, DMSO-d₆, d/ppm): 12.27 (s broad, 2H), 7.83 (d, 1H), 7.78 (dd, 1H), 7.76 (d, 1H), 7.12 (d, 2H), 6.96 (d, 1H), 6.81 (d, 2H), 3.98-3.86 (5H), 3.49 (s, 2H), 2.71-2.60 (3H), 1.97 (pseudo-quint, 2H), 1.78-1.05 (21H), 0.88 (m, 2H).

MS (ESI): m/z=580 (M+H⁺).

### Examples 38 to 42

The compounds of Examples 38 to 42 are prepared in analogy to the process described in Example 30 by reacting the compounds of Examples VI, XIV and XVII with glycine methyl ester hydrochloride or beta-alanine methyl ester hydrochloride.

### Example 38

### Ethyl 4-(3-{4-[4-(cyclohexyloxy)butoxy]phenyl}propoxy)-3-{2-[(3-methoxy-3-oxopropyl)amino]-2-oxoethyl}benzoate

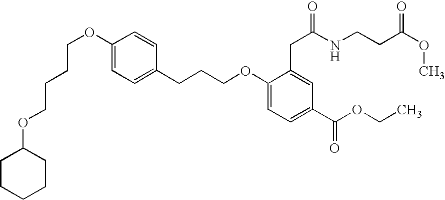

TLC: R_{f}: 0.29 (cyclohexane/ethyl acetate 1:1).

HPLC (Method 2): Rₜ: 5.79 min.

¹H-NMR (300 MHz, DMSO-d₆, d/ppm): 7.98 (t, 1H), 7.82 (dd, 1H), 7.78 (d, 1H), 7.11 (d, 2H), 6.99 (d, 1H), 6.82 (d, 2H), 4.27 (q, 2H), 3.98 (t, 2H), 3.93 (t, 2H), 3.57 (s, 3H), 3.46 (s, 2H), 3.41 (t, 2H), 3.20 (m, 1H), 2.67 (t, 2H), 2.45 (t, 2H), 1.96 (pseudo-quint., 2H), 1.82-1.54 (9H), 1.47 (m, 1H), 1.30 (t, 3H), 1.23-1.16 (6H).

MS (ESI): m/z=598 (M+H⁺).

### Example 39

### Ethyl 4-{3-[4-(3-cyclohexylpropoxy)phenyl]propoxy}-3-{2-[(3-methoxy-3-oxopropyl)amino]-2-oxoethyl}benzoate

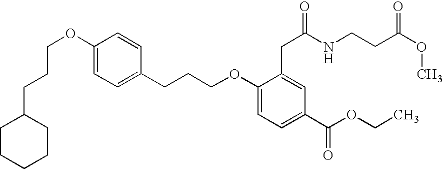

TLC: R_{f}: 0.34 (cyclohexane/ethyl acetate 1:1).

HPLC (Method 1): Rₜ: 6.16 min.

¹H-NMR (300 MHz, DMSO-d₆, d/ppm): 7.98 (t, 1H), 7.82 (dd, 1H), 7.77 (d, 1H), 7.11 (d, 2H), 6.99 (d, 1H), 6.81 (d, 2H), 4.27 (quart, 2H), 3.98 (t, 2H), 3.88 (t, 2H), 3.57 (s, 3H), 3.46 (s, 2H), 3.28 (concealed by H₂O signal, 2H), 2.67 (t, 2H), 2.44 (t, 2H), 1.96 (pseudo-quint, 2H), 1.73-1.58 (7H), 1.30 (t, 3H), 1.31-1.09 (6H), 0.88 (m, 2H).

MS (ESI): m/z=568 (M+H⁺).

### Example 40

### Ethyl 4-(3-{4-[(4-isopropoxybenzyl)oxy]phenyl}propoxy)-3-{2-[(3-methoxy-3-oxopropyl)amino]-2-oxoethyl}benzoate

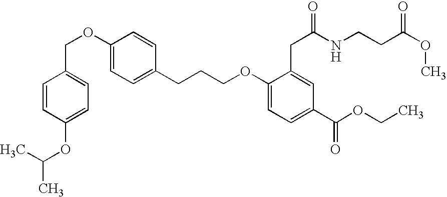

TLC: R_{f}: 0.26 (cyclohexane/ethyl acetate 1:1).

HPLC (Method 1): Rₜ: 5.40 min.

¹H-NMR (300 MHz, DMSO-d₆, d/ppm): 7.98 (t, 1H), 7.82 (dd, 1H), 7.78 (d, 1H), 7.32 (d, 2H), 7.12 (d, 2H), 6.99 (d, 1H), 6.90 (d, 2H), 6.88 (d, 2H), 4.93 (s, 2H), 4.59 (sep, 1H), 4.27 (quart, 2H), 3.98 (t, 2H), 3.56 (s, 3H), 3.47 (s, 2H), 3.28 (concealed by H₂O signal, 2H), 2.68 (t, 2H), 2.45 (t, 2H), 1.97 (pseudo-quint, 2H), 1.30 (t, 3H), 1.23 (d, 6H).

MS (ESI): m/z=592 (M+H⁺).

### Example 41

### Ethyl 4-{3-[4-(3-cyclohexylpropoxy)phenyl]propoxy)}-3-{2-[(2-methoxy-2-oxoethyl)amino]-2-oxoethyl}benzoate

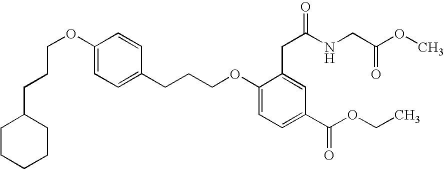

TLC: R_{f}: 0.82 (ethyl acetate/methanol 9:1).

HPLC (Method 2): Rₜ: 6.18 min.

MS (ESI): m/z=554 (M+H⁺).

### Example 42

### Ethyl 4-(3-{4-[(4-isopropoxybenzyl)oxy]phenyl}propoxy)-3-{2-[(2-methoxy-2-oxoethyl)amino]-2-oxoethyl}benzoate

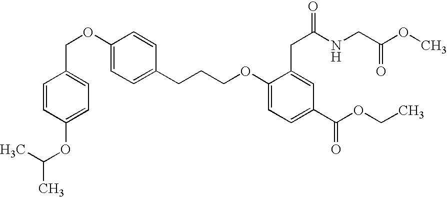

TLC: R_{f}: 0.82 (ethyl acetate/methanol 9:1).

HPLC (Method 2): Rₜ: 5.46 min.

¹H-NMR (300 MHz, DMSO-d₆, d/ppm): 8.32 (t, 1H), 7.83-7.81 (2H), 7.32 (d, 2H), 7.12 (d, 2H), 7.01 (d, 1H), 6.90 (d, 2H), 6.88 (d, 2H), 4.93 (s, 2H), 4.59 (sep, 1H), 4.27 (quart, 2H), 3.99 (t, 2H), 3.83 (d, 2H), 3.59 (s, 3H), 3.54 (s, 2H), 2.67 (t, 2H), 1.98 (pseudo-quint, 2H), 1.30 (t, 3H), 1.23 (d, 6H).

MS (ESI): m/z=578 (M+H⁺).

### Examples 43 to 47

The compounds of Examples 43 to 47 are obtained in analogy to the process described in Example 31 by hydrolyzing the corresponding diesters.

### Example 43

### 3-{2-[(2-Carboxyethyl)amino]-2-oxoethyl}-4-{3-[4-(3-cyclohexylpropoxy)phenyl]propoxy}-benzoic acid

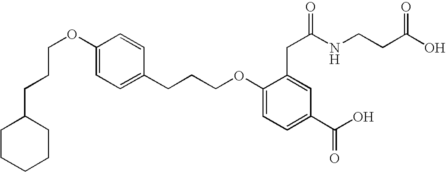

HPLC (Method 2): Rₜ: 5.29 min.

¹H-NMR (400 MHz, DMSO-d₆, d/ppm): 12.37 (s broad, 2H), 7.97 (t, 1H), 7.79 (dd, 1H), 7.75 (d, 1H), 7.11 (d, 2H), 6.97 (d, 1H), 6.81 (d, 2H), 3.95 (t, 2H), 3.88 (t, 2H), 3.45 (s, 2H), 3.24 (quart, 2H), 2.67 (t, 2H), 2.38 (t, 2H), 1.95 (pseudo-quint, 2H), 1.72-1.60 (7H), 1.31-1.07 (6H), 0.87 (m, 2H).

MS (ESI): m/z=526 (M+H⁺).

### Example 44

### 3-{2-[(2-Carboxyethyl)amino]-2-oxoethyl}-4-(3-{4-[(4-isopropoxybenzyl)oxy]phenyl}propoxy)-benzoic acid

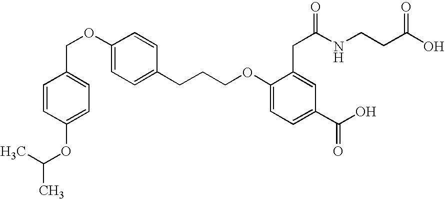

HPLC (Method 2): Rₜ: 4.70 min.

¹H-NMR (400 MHz, CDCl₃, d/ppm): 7.94 (dd, 1H), 7.90 (d, 1H), 7.32 (d, 2H), 7.11 (d, 2H), 6.89 (d, 2H), 6.87 (d, 2H), 6.84 (d, 1H), 6.39 (t, 1H), 4.92 (s, 2H), 4.55 (sep, 1H), 4.02 (t, 2H), 3.56 (s, 2H), 3.43 (quart, 2H), 2.73 (t, 2H), 2.43 (t, 2H), 2.09 (pseudo-quint, 2H), 1.32 (d, 6H).

LC/MS (Method 8): Rₜ: 2.50 min; m/z=549 (M⁺).

### Example 45

### 3-{2-[(Carboxymethyl)amino]-2-oxoethyl}-4-{3-[4-(3-cyclohexylpropoxy)phenyl]propoxy}-benzoic acid

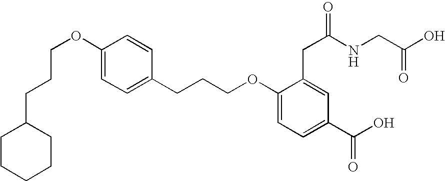

HPLC (Method 2): Rₜ: 5.46 min.

¹H-NMR (300 MHz, DMSO-d₆, d/ppm): 12.48 (s broad, 2H), 8.16 (t, 1H), 7.82-7.78 (2H), 7.11 (d, 2H), 6.97 (d, 1H), 6.81 (d, 2H), 3.97 (t, 2H), 3.89 (t, 2H), 3.74 (d, 2H), 2.67 (t, 2H), 1.97 (pseudo-quint, 2H), 1.72-1.58 (7H), 1.32-1.09 (6H), 0.87 (m, 2H).

MS (ESI): m/z=512 (M+H⁺).

### Example 46

### 3-{2-[(Carboxymethyl)amino]-2-oxoethyl}-4-(3-{4-[(4-isopropoxybenzyl)oxy]phenyl}propoxy)-benzoic acid

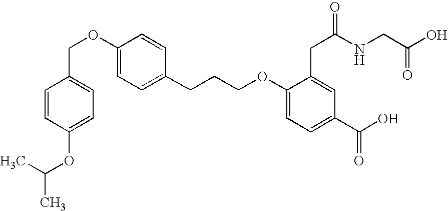

HPLC (Method 2): Rₜ: 4.81 min.

¹H-NMR (300 MHz, DMSO-d₆, d/ppm): 12.48 (s broad, 2H), 8.16 (t, 1H), 7.82-7.80 (2H), 7.32 (d, 2H), 7.12 (d, 2H), 6.98 (d, 1H), 6.90 (d, 2H), 6.88 (d, 2H), 4.93 (s, 2H), 4.59 (sep, 1H), 3.98 (t, 2H), 3.75 (d, 2H), 3.53 (s, 2H), 2.68 (t, 2H), 1.97 (pseudo-quint, 2H), 1.24 (d, 6H).

MS (ESI): m/z=536 (M+H⁺).

### Example 47

### 3-{2-[(Carboxymethyl)amino]-2-oxoethyl}-4-{3-[4-(4-phenoxybutoxy)phenyl]propoxy}benzoic acid

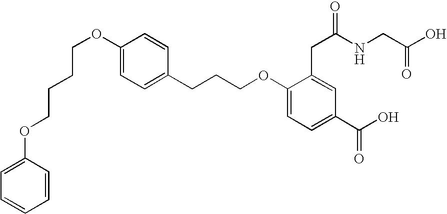

HPLC (Method 2): Rₜ: 4.81 min.

¹H-NMR (300 MHz, DMSO-d₆, d/ppm): 12.47 (s broad, 2H), 8.16 (t, 1H), 7.82-7.80 (2H), 7.29-7.23 (2H), 7.12 (d, 2H), 6.99-6.89 (4H), 6.83 (d, 2H), 4.02-3.98 (6H), 3.76 (d, 2H), 3.53 (s, 2H), 2.68 (t, 2H), 1.97 (pseudo-quint, 2H), 1.85 (4H).

MS (ESI): m/z=536 (M+H⁺).

B. Assessment of the Physiological Activity

Abbreviations:

DMEM Dulbecco's modified Eagle medium

FCS Fetal calf serum

HEPES 4-(2-hydroxyethyl)-1-piperazineethanesulfonic acid

1. Cellular In Vitro Test to Determine the CysLT2 Activity

A recombinant cell line is used to identify antagonists of the human cysteinyl-leukotriene 2 receptor (CysLT2R) and to quantify the activity of the substances described herein. The cell is originally derived from a hamster ovary epithelial cell (Chinese Hamster Ovary, CHO K1, ATCC: American Type Culture Collection, Manassas, Va. 20108, USA). The test cell line constitutively expresses the calcium-sensitive photoprotein aequorin which, after reconstitution with the cofactor coelenterazine, emits light when the cytoplasmic calcium concentration is increased (Rizzuto R, Simpson A W, Brini M, Pozzan T.; Nature 358 (1992) 325-327). The cell is additionally stably transfected with the human CysLT2 receptor (Heise et.al., JBC 275 (2000) 30531-30536). The resulting CysLT2R test cell responds to stimulation of the recombinant CysLT2 receptor (agonists: leukotriene D4 (LTD4) and leukotriene C4 (LTC4)) with an intracellular release of calcium ions, which can be quantified through the resulting aequorin luminescence with a suitable luminometer (Milligan G, Marshall F, Rees S, Trends in Pharmacological Sciences 17 (1996) 235-237). Preincubation with antagonists of the CysLT2 receptor reduces the calcium release induced by the agonists LTD4 and LTC4 and thus the measured amount of light.

Test procedure: The cells are plated out two days before the test in culture medium (DMEM/F12 with Glutamax, Gibco Cat. #61965-026; 10% FCS, Gibco Cat. #10270-106; 1.4 mM sodium pyruvate, Gibco Cat. #11360-039; 1.8 mM sodium bicarbonate, Gibco Cat. #25080-060; 10 mM HEPES, Gibco Cat. #15290-026; now belongs to Invitrogen GmbH, 76131 Karlsruhe) in 384-(or 1536-)well microtiter plates and kept in a cell incubator (96% humidity, 5% v/v CO₂, 37° C.). On the day of the test, the culture medium is replaced by Tyrode solution (in mM: 140 NaCl, 5 KCl, 1 MgCl₂, 2 CaCl₂, 20 glucose, 20 HEPES) which additionally contains the cofactor coelenterazine (50 μM), and the microtiter plate is then incubated for a further 34 hours. 15 minutes after the test substances have been transferred into the wells of the microtiter plate, the resulting light signal is measured after addition of the LTD4 (3×10⁻⁸ M) in the luminometer. The results are shown in Table 1.

2. Cellular In Vitro Test to Determine the CysLT1 Activity

A recombinant cell line is used to identify antagonists of the human cysteinyl-leukotriene 1 receptor (CysLT1R) and to quantify the activity of the substances described herein. The cell is originally derived from a hamster ovary epithelial cell (Chinese Hamster Ovary, CHO K1, ATCC: American Type Culture Collection, Manassas, Va. 20108, USA). The test cell line constitutively expresses the calcium-sensitive photoprotein aequorin which, after reconstitution with the cofactor coelenterazine, emits light when the cytoplasmic calcium concentration is increased (Rizzuto R, Simpson A W, Brini M, Pozzan T.; Nature 358 (1992) 325-327). The cell is additionally stably transfected with the human CysLT1 receptor (Lynch et al., Nature 399 (1999) 789-793). The resulting CysLT1R test cell responds to stimulation of the recombinant CysLT1 receptor (agonist: leukotriene D4 (LTD4)) with an intracellular release of calcium ions, which can be quantified through the resulting aequorin luminescence with a suitable luminometer (Milligan G, Marshall F, Rees S, Trends in Pharmacological Sciences 17 (1996) 235-237). Preincubation with antagonists of the CysLT1 receptor reduces the calcium release induced by the agonist LTD4 and thus the measured amount of light.

Test procedure: The cells are plated out two days before the test in culture medium (DMEM/F12 with Glutamax, Gibco Cat. #61965-026; 10% FCS, Gibco Cat. #10270-106; 1.4 mM sodium pyruvate, Gibco Cat. #11360-039; 1.8 mM sodium bicarbonate, Gibco Cat. #25080-060; 10 mM HEPES, Gibco Cat. #15290-026; now belongs to Invitrogen GmbH, 76131 Karlsruhe) in 384- (or 1536-)well microtiter plates and kept in a cell incubator (96% humidity, 5% v/v CO₂, 37° C.). On the day of the test, the culture medium is replaced by Tyrode solution (in mM: 140 NaCl, 5 KCl, 1 MgCl₂, 2 CaCl₂, 20 glucose, 20 HEPES) which additionally contains the cofactor coelenterazine (50 μM), and the microtiter plate is then incubated for a further 3-4 hours. 15 minutes after the test substances have been transferred into the wells of the microtiter plate, the resulting light signal is measured after addition of the LID4 (3×10⁻⁹ M) in the luminometer. The results are shown in Table 1.

TABLE 1
CysLT1 activity/CysLT2 activity comparison

| | CysLT1 | CysLT2 |
|---|---|---|
| Example No. | IC₅₀ (nM) | IC₅₀ (nM) |
| 13 | >10000 | 140 |
| 25 | >10000 | 45 |
| 31 | >10000 | 45 |
| 33 | >3000 | 70 |
| 45 | >3000 | 75 |

3. In Vivo Test to Detect the Cardiovascular Effect: Langendorff Guinea Pig Heart

The heart is removed after opening the chest cavity of anesthetized guinea pigs and is introduced into a conventional Langendorff apparatus. The coronary arteries are subjected to a constant volume (10 ml/min) perfusion, and the perfusion pressure arising thereby is recorded via an appropriate pressure transducer. A decrease in the perfusion pressure in this arrangement corresponds to a relaxation of the coronary arteries. At the same time, the pressure developed by the heart during each contraction (the left ventricular pressure) is measured by a balloon introduced into the left ventricle, and a further pressure transducer. The rate at which the isolated heart beats is found by calculation from the number of contractions per unit time. To detect the effect of CysLT2 receptors antagonists, the perfusion with the agonist LTC4 (10⁻⁸ M) is started 15 minutes before addition of increasing concentrations of the test substance (10⁻⁸ to 10⁻⁶ M).

C. Exemplary Embodiments of Pharmaceutical Compositions

The compounds of the invention can be converted into pharmaceutical preparations in the following way:

Tablets:

Composition:

100 mg of the compound of Example 11, 50 mg of lactose (monohydrate), 50 mg of microcrystalline cellulose, 10 mg of polyvinylpyrrolidone (PVP) (from BASF, Ludwigshafen, Germany), 10 mg of crosslinked Na carboxymethylcellulose and 2 mg of magnesium stearate.

Tablet weight 222 mg. Diameter 8 mm, radius of curvature 12 mm.

Production:

The mixture of active ingredient, lactose and cellulose is granulated with a 5% strength solution (m/m) of the PVP in water. The granules are dried and then mixed with the crosslinked Na carboxymethylcellulose and the magnesium stearate for 5 minutes. This mixture is compressed in a conventional tablet press (see above for tablet format). As a guideline, a compressive force of 15 kN is used for the compression.

Suspension Which can be Administered Orally:

Composition:

1000 mg of the compound of Example 11, 1000 mg of ethanol (96%), 400 mg of xanthan gum (from FMC, Pennsylvania, USA) and 97.6 g of water. 10 g of oral suspension correspond to a single dose of 100 mg of the compound of the invention.

Production:

The xanthan gum is suspended in ethanol, and the active ingredient is added to the suspension. The water is added while stirring. The mixture is stirred for about 6 h until the xanthan gum has finished swelling.

Solution Which can be Administered Orally:

Composition

500 mg of the compound of Example 11, 2.5 g of polysorbate and 97 g of polyethylene glycol 400. 20 g of oral solution correspond to a single dose of 100 mg of the compound of the invention.

Production

The active ingredient is suspended by stirring in the mixture of polyethylene glycol and polysorbate. The stirring process is continued until the active ingredient has completely dissolved.

Solution Which can be Administered Intravenously:

The active ingredient is dissolved in a concentration below the saturation solubility in a physiologically tolerated solvent (see Examples). The solution is sterilized by filtration and dispensed into sterile and pyrogen-free injection/infusion containers.

Composition I:

100 mg of the compound of Example 11, 15 g of polyethylene glycol 400 and 250 g of a 2% strength aqueous sodium bicarbonate solution for injections.

Production:

The compound of Example 11 is dissolved together with polyethylene glycol 400 by stirring in the 2% strength aqueous sodium bicarbonate solution. The solution is sterilized by filtration (pore diameter 0.22 μm) and dispensed under aseptic conditions into heat-sterilized infusion bottles. The latter are closed with infusion stoppers and crimped caps.

Composition II:

100 mg of the compound of Example 11 and 250 ml of an aqueous solution of 0.31 g of anhydrous citric acid and 5.66 g of sodium monohydrogen phosphate dihydrate.

Production:

The compound of Example 11 is dissolved by stirring in the aqueous solution. The solution is sterilized by filtration and dispensed under aseptic conditions into sterile and pyrogen-free injection/infusion containers.

Composition III:

100 mg of the compound of Example 11 and 250 ml of an aqueous solution of 0.044 g of anhydrous citric acid, 0.81 g of sodium monohydrogen phosphate dihydrate and 1.87 g of sodium chloride.

Production:

The compound of Example 11 is dissolved by stirring in the aqueous solution. The solution is sterilized by filtration and dispensed under aseptic conditions into sterile and pyrogen-free injection/infusion containers.

1. A compound of the formula 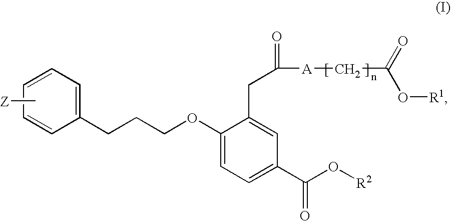
in which
A is a 4- to 7-membered nitrogen-containing saturated heterocycle which is bonded via the nitrogen atom to the keto group,
or
is a radical 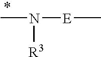
in which
E is (C₄-C₇)-cycloalkanediyl or (C₁-C₆)-alkanediyl,
R³ is hydrogen or methyl and
* is the point of linkage to the keto group,
n is 0, 1 or 2,
R¹ is hydrogen or (C₁-C₆)-alkyl,
R² is hydrogen or (C₁-C₆)-alkyl,
and
Z is located in the meta or para position and
is a radical *-G-L-M-R⁴
in which
G is O,
L is (C₁-C₆)-alkanediyl,
M is a bond or O,
R⁴ is (C₆-C₁₀)-aryl, biphenyl, phenoxyphenyl, benzyloxyphenyl, (E)-phenylvinylphenyl, 2-phenylethylphenyl, tetrahydronaphthyl, benzyl, heteroaryl, 5- to 10-membered heterocyclyl, (C₃-C₇)-cycloalkyl or (C₃-C₇)-cycloalkylmethyl, where aryl, biphenyl, phenoxyphenyl, benzyloxyphenyl, (E)-phenylvinylphenyl, 2-phenylethylphenyl, tetrahydronaphthyl, benzyl, heteroaryl, heterocyclyl, cycloalkyl and cycloalkylmethyl in turn may be substituted up to three times independently of one another by halogen, cyano, nitro, trifluoromethyl, trifluoromethoxy, (C₁-C₆)-alkyl, (C₁-C₆)-alkoxy, (C₂-C₆)-alkenyl, (C₃-C₇)-cycloalkyl, (C₃-C₇)-cycloalkylmethoxy, (C₅-C₇)-cycloalkenyl, (C₃-C₇)-cycloalkoxy or (C₅-C₇)-cycloalkenyloxy, and
* is the point of linkage to the phenyl ring,
or a salt, solvate, or solvate of a salt thereof.

2. The compound as claimed in claim 1, characterized in that
A is a 4- to 6-membered nitrogen-containing saturated heterocycle which is bonded via the nitrogen atom to the keto group,
or
is a radical 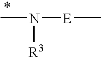
in which
E is cyclopentanediyl, cyclohexanediyl, methylene, ethane-1,2-diyl or propane-1,3-diyl,
R³ is hydrogen, and
* is the point of linkage to the keto group,
n is 0 or 1,
R¹ is hydrogen,
R² is hydrogen,
and
Z is located in the meta or para position and
is a radical *-G-L-M-R⁴
in which
G is O,
L is (C₁-C₄)-alkanediyl,
M is a bond or O,
R⁴ is phenyl, naphthyl, biphenyl, phenoxyphenyl, benzyloxyphenyl, (E)-phenylvinylphenyl, 2-phenylethylphenyl, tetrahydronaphthyl, benzyl, 1,3-dioxanyl, 1,4-dioxanyl, dimethyl-1,3-dioxanyl, tetrahydro-2H-pyranyl, (C₃-C₇)-cycloalkyl or (C₃-C₇)-cycloalkylmethyl, where phenyl, naphthyl, biphenyl, phenoxyphenyl, benzyloxyphenyl, (E)-phenylvinylphenyl, 2-phenylethylphenyl, tetrahydronaphthyl, benzyl, cycloalkyl and cycloalkylmethyl in turn may be substituted up to three times independently of one another by halogen, cyano, nitro, trifluoromethyl, trifluoromethoxy, (C₁-C₆)-alkyl, (C₁-C₆)-alkoxy, (C₃-C₇)-cycloalkyl, (C₃-C₇)-cycloalkylmethoxy or (C₃-C₇)-cycloalkoxy, and
* is the point of linkage to the phenyl ring,
or a salt, solvate, or solvate of a salt thereof.

3. The compound as claimed in claim 1, characterized in that
A is acetidine, pyrrolidine or piperidine which is bonded via the nitrogen atom to the keto group,
or
is a radical 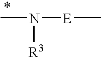
in which
E is cyclopentanediyl, cyclohexanediyl, methylene, ethane-1,2-diyl or propane-1,3-diyl,
R³ is hydrogen, and
* is the point of linkage to the keto group,
n is 0 or 1,
R¹ is hydrogen,
R² is hydrogen,
and
Z is located in the para position and
is a radical *-G-L-M-R⁴
in which
G is O,
L is methylene, propane-1,3-diyl or butane-1,4-diyl,
M is a bond or O,
R⁴ is phenyl, 4-biphenyl, 4-phenoxyphenyl, 4-benzyloxyphenyl, 1,2,3,4-tetrahydronaphth-6-yl, 5,5-dimethyl-1,3-dioxan-2-yl or cyclohexyl, where phenyl in turn may be substituted by halogen, trifluoromethoxy, (C₃-C₄)-alkyl, (C₃-C₄)-alkoxy, cyclopentyl, cyclohexyl or (C₃-C₆)-cycloalkyl-methoxy, and
* is the point of linkage to the phenyl ring,
or a salt, solvate, or solvate of a salt thereof.

4. The compound as claimed in claim 1, characterized in that
A-[CH₂]ₙ—CO₂R¹ is a radical 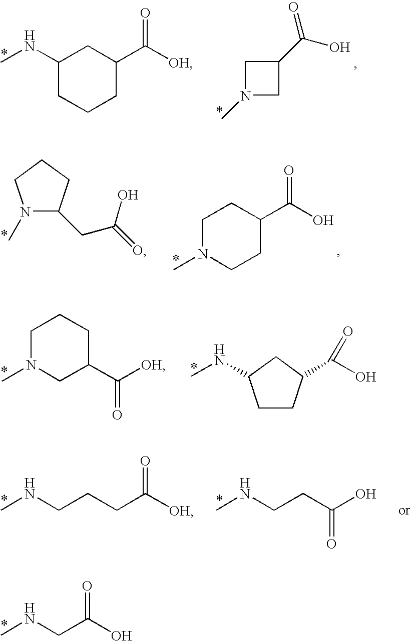
in which
* is the point of linkage to the keto group,
R² is hydrogen,
and
Z is located in the para position and is
a radical *—O—CH₂—R⁴,
in which
R⁴ is phenyl, 4-biphenyl, 4-phenoxyphenyl, 4-benzyloxyphenyl or 1,2,3,4-tetrahydronaphth-6-yl, where phenyl in turn may be substituted by trifluoromethoxy, n-propyl, n-butyl, tert-butyl, n-propyloxy, isopropyloxy, isobutyloxy, cyclohexyl or cyclopropylmethoxy, and
* is the point of linkage to the phenyl ring,
or
is a radical *—O—CH₂—CH₂—CH₂—R⁴
in which
R⁴ is 4-chlorophenyl, 5,5-dimethyl-1,3-dioxan-2-yl or cyclohexyl, and
* is the point of linkage to the phenyl ring,
or
is a radical *—O—CH₂—CH₂—CH₂—CH₂—O—R⁴
in which
R⁴ is phenyl or cyclohexyl, and
* is the point of linkage to the phenyl ring,
or a salt, solvate, or solvate of a salt thereof.

5. The compound as claimed in claim 1, characterized in that
A is acetidine, pyrrolidine or piperidine which is bonded via the nitrogen atom to the keto group,
or
is a radical 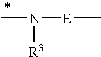
in which
E is cyclopentanediyl, cyclohexanediyl, methylene, ethane-1,2-diyl or propane-1,3-diyl,
R³ is hydrogen, and
* is the point of linkage to the keto group,
n is 0 or 1,
R¹ is hydrogen,
R² is hydrogen,
and
Z is located in the para position and is a radical 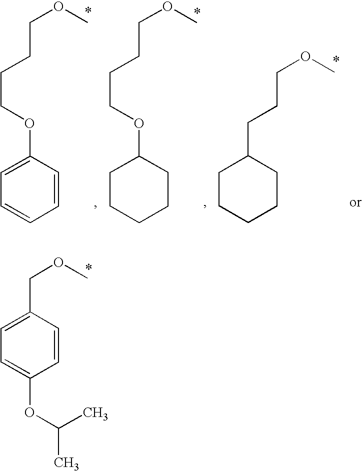
in which
* is the point of linkage to the phenyl ring,
or a salt, solvate, or solvate of a salt thereof.

6. The compound as claimed in claim 1, characterized in that
A-[CH₂]ₙ—CO₂R¹ is a radical 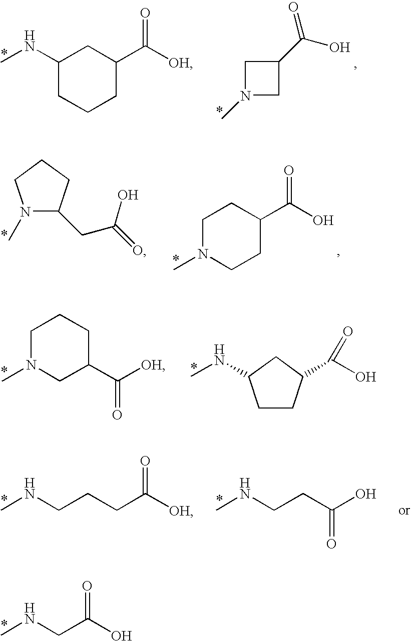
in which
* is the point of linkage to the keto group,
R² is hydrogen,
and
Z is located in the para position and is a radical 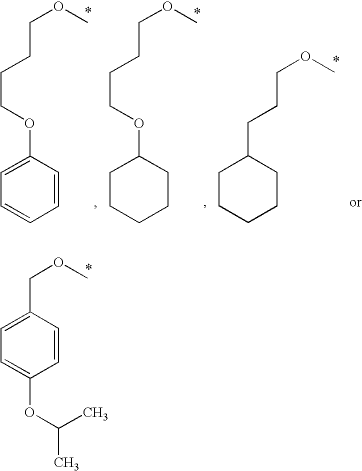
in which
* is the point of linkage to the phenyl ring,
or a salt, solvate, or solvate of a salt thereof.

7. A process for preparing a compound of the formula (I) as defined in claim 1 or one of its salts, solvates or solvates of its salts, characterized in that
either
(A) a compound of the formula (II) 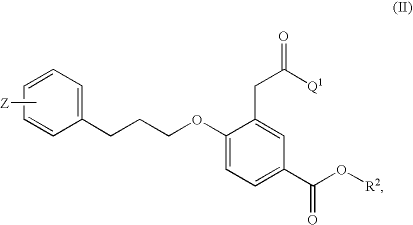
in which
R² is (C₁-C₆)-alkyl,
Q¹ is hydroxy or chlorine, and
Z has the meaning indicated in claim 1,
is reacted with a compound of the formula (III) 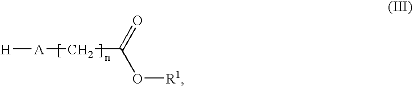
in which
R¹ is (C₁-C₆)-alkyl, and
n and A have the meaning indicated in claim 1,
or
(B) the two ester groups —C(O)OR¹ and —C(O)OR² in a compound prepared by process step (A) are hydrolyzed.

8. (canceled)

9. A pharmaceutical composition comprising a compound as defined claim 1 in combination with an inert, non-toxic, pharmaceutically suitable excipient.

10. A pharmaceutical composition comprising a compound as defined in claim 1 in combination with a further active ingredient.

11. A method for the treatment and/or prophylaxis of cardiovascular disorders comprising administering an effective amount of a compound as defined in claim 1.

12. (canceled)

13. (canceled)

14. The use method as claimed in claim 11 wherein the cardiovascular disorder is unstable angina pectoris or myocardial infarction.
